(19) United States
(12) Patent Application Publication
(10) Pub. No.: US 2013/0109048 A1
(43) Pub. Date: May 2, 2013

(54) METHOD FOR IN-VITRO MONITORING OF NEURONAL DISORDERS AND USE THEREOF

(71) Applicants: Michele Giugliano, Bern (CH); Ruth Luthi-Carter, Leicester (GB); Henry Markram, Lausanne (CH); Luca Gambazzi, Rancate (CH); Ozgün Gökce, Hatay/Izmir (TR)

(72) Inventors: Michele Giugliano, Bern (CH); Ruth Luthi-Carter, Leicester (GB); Henry Markram, Lausanne (CH); Luca Gambazzi, Rancate (CH); Ozgün Gökce, Hatay/Izmir (TR)

(73) Assignee: ECOLE POLYTECHNIQUE FEDERALE DE LAUSANNE (EPFL), LAUSANNE (CH)

(21) Appl. No.: 13/809,315

(22) PCT Filed: Jul. 11, 2011
(86) PCT No.: PCT/IB11/53073

§ 371 Date: Jan. 9, 2013

(30) Foreign Application Priority Data
Jul. 9, 2010 (CH) 01124/10

(51) Int. Cl. C12Q 1/02

(52) U.S. Cl. CPC C12Q 1/025
USPC 435/29; 435/325

(57) ABSTRACT

The present invention relates to methods and neuronal cellular preparations allowing monitoring intracellular and transcellular molecular events on both short and long timescales in an ex vivo neuronal network of intact post-mitotic neurons. In particular, the invention relates to methods, kits, genetically engineered neuronal cells, preparations and uses thereof allowing the ex vivo monitoring of early neuronal disease-related changes in neuronal network behavior.

### FIELD OF THE INVENTION

The present invention pertains generally to the fields of drug discovery and neuroscience, and more particularly to methods and cellular preparations for the detection and characterization of neurologically active substances based on their effect on cell electrophysiology under conditions mimicking those observed in vivo.

### BACKGROUND OF THE INVENTION

Diseases of the central nervous system (CNS) are some of the most prevalent and devastating but yet poorly treatable so far. Very few truly innovative CNS drugs have been approved in recent years. According to a recent survey, CNS drug candidates entering clinical development have a considerably lower probability (7%) of reaching the market in comparison to other therapeutic areas (15%), suggesting that there is a considerable need to improve/enhance CNS drug discovery strategies. CNS drugs fail mainly due to the sheer complexity of the brain, an organ that is difficult to model (Pangalos, 2007, Nat. Rev. Drug Discov., 6:521).

One of the major problems with CNS drug screening today is that the predominant approaches used to carry out CNS drug discovery use systems and techniques that oversimplify the neurobiological problem. Purely molecular approaches such as fluorescence (Fluorescent Imaging Plate Reader, FLIPPR, Molecular Devices) and planar patch-clamp based techniques such as IonWorks HT (Molecular Devices) carry out drug screening against isolated, heterologously expressed ion channels, which often identify drug candidates whose higher-order impact cannot necessarily be inferred from their effects on individual conductances (Dunlop, 2008, Nat. Rev. Drug Discov., 7:358; Pangalos, 2007, above).

In addition, cell-based assays typically use transformed cell lines with non-neuronal or poor neuron-like phenotypes. Moreover, transfection-based methods do not achieve uniform expression levels in neurons and are problematic for examining population-based behaviours.

Further, it not possible to carry out large-scale, long-term assessment of the neuronal electrical activity in such disease models, which therefore present a further serious shortcoming: those assays do not permit the study of chronic effects or toxicity as they rely on short-term (e.g. 12 h, 24 h and 48 h) application or expression of disease-causing substances.

In addition, known CNS assays do not allow the monitoring of effects of CNS-targeted therapies through their impact on synaptic physiology, as synaptic activity is only modelled accurately in neuronal cells capable of forming such intricately complex structural and molecular coupling. However, synaptic plasticity has been identified as a common important feature in animal models of various neurodegenerative disorders (Bagetta et al., 2010, Biochem. Soc. Trans., Apr; 38(2):493-7; Nimmrich and Ebert, 2009, Rev. Neurosci., 20:1-12; Di Filippo et al., 2007, Curr. Opin. Pharmacol., Feb; 7(1):106-11).

Another major limitation of known methods for assessing the deterioration of neuronal function is the inability to detect the very earliest stages of the process, where recovery of a function may most easily be achieved. The current state-of-the-art protocols for investigating and quantifying the neuroprotective effects of candidate therapies typically involve the counting of surviving cells. Thus, these assays are only able to report late downstream effects of the detrimental process, i.e. consequent cell death. Moreover, substances revealed as positives (“hits”) by such screening assays conducted in non-neuronal cells are extremely poor predictors of the effects of those substances in neuronal cells or the brain.

Therefore, there is an emerging need for developing new methods and tools for carrying out pharmacological evaluations of CNS active substances with sufficient throughput in such higher-order, truly neuronal systems (Dunlop, 2008, above).

### SUMMARY OF THE INVENTION

The present invention is directed to methods and neuronal cellular preparations allowing monitoring of intracellular and transcellular molecular events on both short and long timescales in an ex vivo neuronal network of intact post-mitotic neurons. In particular, the invention relates to the unexpected findings that combination of genetically engineered cortical neurons modeling a neuronal disorder such as Huntington's disease (HD) and the ex vivo recording of the electrophysiological activity of those cells, in particular through extracellular multielectrode array (MEA) in vitro recordings, allows the observation of early disease-related changes in neuronal network behavior.

A first aspect of the invention provides an ex vivo method for assaying a neuroactive substance comprising:
(i) Providing a neuronal cell culture sample comprising genetically engineered neuronal cells wherein neuronal cells are engineered to express a neurodegeneratively active protein;
(ii) Comparing at least one electrophysiological response parameter measured simultaneously at a plurality of regions in said neuronal cell culture sample when contacted with a candidate substance or a candidate substance composition with a baseline electrophysiological response parameter of said regions;
(iii) Determining the difference between said electrophysiological response parameter and said baseline electrophysiological response parameter;
(iv) Detecting the presence or absence of a neuroactive substance in said candidate substance or candidate substance composition based upon the difference determined under step (iii) and/or detecting a neuronal adverse effect of said candidate substance or candidate substance composition based upon the difference determined under step (iii).

A second aspect of the invention provides a kit for electrophysiological detection of a neuroactive substance, the kit comprising genetically engineered neuronal cells or a composition thereof or neuronal cells together with vectors to genetically engineer neuronal cells, wherein the genetically engineered neuronal cells or the neuronal cells once transduced with the said vectors express a neurodegeneratively active protein.

A third aspect of the invention provides a genetically engineered neuronal cell according to the invention for the electrophysiological detection of a neuroactive substance wherein the neuronal cells are genetically engineered to express a neurodegeneratively active protein.

A fourth aspect of the invention provides a use of an engineered neuronal cell according to the invention in the preparation of a composition for the electrophysiological detection of a neuroactive substance.

A fifth aspect of the invention provides isolated neuronal cells transduced by a lentiviral vector according to the invention.

A sixth aspect of the invention provides an isolated neuronal cell composition comprising at least one cell according to the invention.

Other features and advantages of the invention will be apparent from the detailed description, figures and sequence listings.

### DESCRIPTION OF THE FIGURES

FIG. 1 shows a schematic representation of lentiviral constructs for ex vivo cortical cell model of HD according to the invention and electrophysiological recordings of spontaneously active ex vivo cortical networks as described in Example 1. (A) Lentiviral expression vectors encoding wild-type (18Q) or mutated (82Q) htt fragments under the control of a tetracycline-regulated element promoter containing the Tet-response element (TRE) with seven direct repeats of the bacterial tetO tetracycline operator sequence upstream of a minimal Cytomegalovirus promoter (Pmin) (SIN-TRE-htt171) and encoding the tetracycline-controlled transactivator tTA1 (i.e. tetracycline repressor tetR fused to four copies (4F) of the minimal transcriptional activation domain of VP16) under the control of the mouse PGK (phosphoglycerate kinase) promoter as described in Example 1. Both vectors are self-inactivating (SIN) and contain a posttranscriptional woodchuck hepatitis B virus regulatory element (WPRE). (B) Scheme depicting the experimental timeline. (C) Counts of NeuN-positive neuronal nuclei in the cultures expressing htt171-82Q. Data are presented as percent of control (htt171-18Q) for each time point. Bars represent mean values±SEM. * p<0.05. (D) Cultures on glass arrays of 60 substrate planar microelectrodes (left panel). Each electrode independently detects extracellular action potentials of nearby neurons (right panel): (top) sample voltage recordings from 10 independent electrodes, displaying as a function of time the electric potential recorded extracellularly during 1 second. Fast deflection of larger amplitude, compared to the rest of the traces, represents the effect of emission of nerve impulse by neurons growing in close proximity to the substrate electrodes (left panel). A zoomed area of a sample voltage recording is presented in the bottom trace, enlarging both the horizontal and the vertical scale to reveal the shape of the detected nerve impulse (bottom). (E) Electrophysiological recordings revealing spontaneous activity consisting of asynchronous firing and rare bursts of events (zoomed area) synchronized across MEA electrodes.

FIG. 2 shows the voltage recordings obtained at each of the individual electrode of the MEA, obtained from cultured neurons during long-term experiments. The electrical signatures (i.e. “spikes”) of the neuronal excitable physiology reported in FIG. 1E have been graphically represented in a “raster plot” (A—top), where the time of occurrence of each nerve impulse (i.e. a “spike”, see FIG. 1E) has been indicated by a small dot. Such a representation of the raw electrophysiological data recorded experimentally has been carried out in control cultures, treated by htt171-18Q (n=11), and in sister cultures, treated by htt171-82Q to display the HD (n=12), as described in Example 1. (A) The electrical activity displayed in control and disease cultures, u) detected at 14 days in vitro, is characterized by episodes of synchronized electrical activity, apparent as vertical stripes when the spike-time histograms (STHs) (A—bottom) are computed and graphically shown. These vertical stripes are called Population Bursts (PBs). PBs had similar durations and distributions across the entire MEA surface and they can be studied in details by statistical analysis. In (B), the number of spikes recorded during a period of 30 min of continuous recording is represented as bars, whose colors indicate the experiments they refer to (i.e. control or disease treated cultures). Other quantities are represented, such as the number of PB, the average time interval between successive PBs (i.e. IBI), the average duration of each PB and the average number of spikes occurred in the time interval between (i.e. inter-burst) two successive PBs. In (C) and (D), the same quantities (i.e. the IBI and the PB duration) that have been analyzed and compared in (B) are displayed not as average values but as cumulative distributions, which is a way to count and compare individual quantities and not only their average values. In (E), each point corresponds to an individual PB, recorded during an experimental session lasting 30 minutes: the position of the dot in the graph allows visualization of the number of the spikes that composed that PB (i.e. the horizontal axis) and the duration of the PB (i.e. the vertical axis). Different colors refer to control and disease cultures. In (F), an alternative analysis was carried out and displayed, showing the cumulative distribution of the numerical values of an index, the cross-correlation, which measures the similarity between subsequent spikes recorded at two generic electrodes of the same MEAs. Larger values (e.g. 0.6-0.8) mean larger similarity of the time at which spikes were recorded from distinct electrodes, being an indication on how much coordinated or synchronised the electrical activity is. In (G), the average similarity between series of spikes recorded at generic pairs of electrodes of the same MEAs (as in F) has represented for electrode pairs, chosen among those available in the MEA with increasing distances—from 100 micrometers to more than 1000 micrometers.

Whenever an asterisk (*) is indicated in the panels above, it means that significant differences between control and disease culture have been reported. This significance is established by statistical methods, ensuring that only in 5% of the cases the experimental results could be attributed to chance, instead of to the effect of the disease.

FIG. 3 shows the effect of applying the substance BDNF on the average number of PBs, obtained in 30 minutes lasting experiments and by 2 week-old neuronal cultures in vitro, as described in Example 1, 5 hours prior to (left), 45 min after (middle) and 24 h after (right) K252a addition (n=3 control MEAs without K252a addition, n=3 MEAs with 10 nM K252a and n=3 MEAs with 100 nM K252a.

FIG. 4 repeats the same analysis as FIG. 2, comparing not only the control to the disease cultures, but also studying how the treatment by BDNF rescued the disease culture. Control htt171-18Q treated cultures (n=2), and htt171-82Q treated sister cultures were treated (n=6) or not treated (n=6) with BDNF (50 ng/ml, added twice weekly) after 23 days in vitro, as described in Example 1. In (A), the number of spikes recorded during a period of 30 min of continuous recording is represented as bars, whose colors indicate the experiments they refer to (i.e. control, disease treated cultures, and disease treated cultures exposed to BDNF). Are also represented, the number of PB, the average time interval between successive PBs (i.e. IBI), the average duration of each PB and the average number of spikes occurred in the time interval between (i.e. inter-burst) two successive PBs. In (B) and (C), the IBI and the PB duration analyzed and compared in (A) are displayed as cumulative distributions, a way to count and compare individual quantities and not only their average values. In (D), each point corresponds to an individual PB, recorded during an experimental session lasting 30 minutes: the position of the dot in the graph allows visualization of the number of the spikes that composed that PB (i.e. the horizontal axis) and the duration of the PB (i.e. the vertical axis). Different colors refers to control and disease culture. In (E), the results from the analysis of epifluorescence microscope photographs were displayed to show how many subcellular structures could be identified within the same image field of view, comparing control, disease cultures and disease cultures exposed to BDNF. Such subcellular structures, indirectly visible from the accumulation of a immunostained protein (i.e. PSD95), are the synapses. Whenever one or two asterisk (*) is indicated in the panels above, it means that significant differences—compared to control—have been reported. This significance is established by statistical methods, ensuring that only in less than 5% (*) or in less than 1% of the cases, the experimental results could be attributed to chance, instead of to the effect of the disease.

FIG. 5 shows sequences of SEQ ID NO: 1 to 14 used in the context of the invention.

### DETAILED DESCRIPTION OF THE INVENTION

The term “spike” refers to an electrical pulse or action potential propagated by neurons.

The term “spike train” refers to an action potential sequence.

The term “burst” relates to high frequency spike episodes. Bursting is a dynamic state where a neuron repeatedly fires discrete groups or bursts of spikes. Each such burst is followed by a period of quiescence before the next burst occurs.

The term “firing rate” refers to the average number of spikes per unit of time.

The term “epoch” refers to a time interval characterized by the co-occurrence of spikes, synchronized across several electrodes of the multielectrode array.

The term “waveform” refers to the analog electric voltage recording across time, captured around the peak of a spike and therefore enabling one to create an average across many individual waveforms.

The term “baseline electrophysiological parameter” refers to an electrophysiological parameter that is measured prior to contact of neuronal cell sample with a candidate substance. Examples of baseline parameters are action potential characteristics such as frequency, amplitude, shape, spike kinetics, number of spikes, number of population bursts, temporal correlations between action potentials measured between any possible pair of electrodes.

The term “electrophysiological response parameter” refers to an electrophysiological parameter that is measured after contact of a neuronal cell sample with a candidate substance. Examples of response parameters are changes in action potential characteristics such as frequency, amplitude, shape, spike kinetics, number of spikes, number of population bursts, temporal correlations between action potentials measured between any possible pair of electrodes.

The term “neuronal cells” refers to isolated primary neuronal cells such as cortical or hippocampal neurons, which have been displaced and dissociated from the nerve tissue they were composing and which may optionally be cultured together with accompanying astroctytes. These cells can be extracted from brain tissue obtained from rat embryos or from newborn rats. These cells may be preserved or stored at 2-8° C. in suitable medium conditions such as described in Kawamoto and Barrett Brain Research 384(1):84-93. In a particular embodiment, when present in a kit of the invention, these cells may be preserved for example by placing those cells or small pieces of brain tissue (<8 mm³) into a medium of pH 7.3, containing 50 mM K⁺, 20 mM Na⁺, 25 mM PO₄²⁻, 20 mM lactic acid, 5 mM glucose, and low Ca²⁺ (<0.1 mM), made isotonic by adding sorbitol. These cells can be stored at 2-8° C. for more than a week. With addition of 10% DMSO, these cells can be stored frozen at −70 to −90° C. for up to about 3 months.

The term “neuronal cell sample” refers to neuronal cells according to the invention within a suitable neuronal cell culture medium.

The term “neurodegenerative disease or disorder” comprises a disease or a state characterized by a central nervous system (CNS) degeneration or alteration, especially in neurons, such as Alzheimer's disease (AD), Parkinson's disease (PD), Dementia with Lewy bodies (DLB), Frontotemporal dementia (FTD), Huntington's disease (HD) and amyotrophic lateral sclerosis (ALS).

The term “neurodegeneratively active protein” comprises proteins causatively involved in a neurodegenerative disease or disorder. In a particular embodiment, the neurodegeneratively active protein is a mutant of huntingtin protein (SEQ ID NO: 1) or a variant or a fragment thereof (e.g. a mutant of huntingtin protein fragment consisting of the first 171 amino acids of the huntingtin protein carrying 82Q glutamines (SEQ ID NO: 2). In another embodiment, the neurodegeneratively active protein is a protein selected from full-length amyloid precursor protein (APP) (SEQ ID NO: 3) or a variant thereof (e.g. APP with missense mutation KM/670/671/NL or missense mutation E693G or missense mutation V717F) or a fragment thereof, microtubule-associated protein tau (MAPT) (SEQ ID NO: 4) or a variant thereof (e.g. Missense MAPT mutation L618P) or a fragment thereof, full-length presenilin 1 (PSEN1) (SEQ ID NO: 5), or a variant thereof (e.g. PSEN1 missense mutation V82L or missense mutation V96F or deletion IM 83/84) or a fragment thereof, granulin (GRN) (SEQ ID NO: 6) or a variant thereof or a fragment thereof, alpha-synuclein (SNCA) isoform 1 (SEQ ID NO: 7), isoform 2-4 (SEQ ID NO: 8), isoform 2-5 (SEQ ID NO: 9) or a variant thereof or a fragment thereof, leucine-rich repeat kinase 2 (LRRK2) (SEQ ID NO: 10) or a variant thereof (e.g. missense mutation G2019S) or a fragment thereof, ATPase type 13A2 (ATP13A2) (SEQ ID NO: 11) or a variant thereof (e.g. missense mutation G504R) or a fragment thereof, superoxide dismutase 1 (SOD1) (SEQ ID NO: 12) or a variant thereof (e.g. missense mutation G93A) or a fragment thereof and dynactin 1 (DCTN1) (SEQ ID NO: 13) or a variant thereof (e.g. missense mutation M571T) or a fragment thereof.

The term “candidate substance” refers to any substance whose effect on a neuronal cell composition according to the invention, one is attempting to determine. It includes substances potentially neuroactive or substances known to be neuroactive for which one is attempting to test potential neuronal adverse or toxic effects. A candidate substance includes, but is not limited to, drugs, proteins, peptides, carbohydrates, nucleic acids, lipids, natural products, peptidomimetics, antibodies, small molecules and the like.

The term “candidate substance composition” refers to any composition comprising a candidate substance.

The term “neuroactive substance” includes a substance which is able to prevent, repress or treat neuronal dysfunctions, in particular those characterizing a neurodegenerative disease or disorder, including those characterizing early stage of a neurodegenerative disease or disorder, as measured by electrophysiological detection on genetically engineered neuronal cells according to the invention. Alternatively, electrophysiological detection can be accompanied or complemented by the detection of other parameters, such as the morphology and the physiology of neurons, obtained for instance by microscopy. These substances include, but are not limited to, drugs, proteins, peptides, carbohydrates, nucleic acids, lipids, natural products, peptidomimetics, antibodies, small molecules and the like.

The term “adverse effect” includes an effect at the neuronal level including a noxious neuronal effect such as an abnormal increase or decrease in electrical activity parameters or abnormalities of neuronal morphology, as measured by electrophysiological detection on genetically engineered neuronal cells according to the invention, optionally accompanied or complemented by the detection of other parameters, such as the morphology and the physiology of neurons, obtained for instance by microscopy.

The term “treat” or “treating” refers to the capacity of obtaining a desired physiological effect. The effect may be prophylactic in terms of preventing or partially preventing a neuronal dysfunction, and/or may be therapeutic in terms of a partial or complete cure of a neuronal dysfunction by reversing an existing neuronal dysfunction. The term “treatment” as used herein covers: (a) preventing the neuronal dysfunction from occurring in a neuronal cell sample which may be predisposed to exhibit neuronal dysfunction but has not yet been diagnosed as having it; (b) inhibiting the neuronal u) dysfunction, i.e., limiting or arresting its development; or relieving the neuronal dysfunction, i.e., causing regression of the neuronal dysfunction such as improvement or remediation of neural damage.

The term “variant” means a polypeptide or a protein substantially homologous to the native sequence, but which has an amino acid sequence different from that of native sequence because of one or more deletions, insertions or substitutions. Typically, substantially homologous means a variant amino acid sequence that is at least 80%, at least 85%, at least 90%, at least 95%, at least 96%, at least 97%, at least 98% or at least 99% identical to the native amino acid sequences, as disclosed above. The percent identity of two amino acid or two nucleic acid sequences can be determined by visual inspection and/or mathematical calculation, or more easily by comparing sequence information using known computer program used for sequence comparison such as Clustal package version 1.83. A variant may accommodate one or more tagging sequences or other potentially desirable modifications.

The term “fragment” means a polypeptide or proteins or a variant thereof which has an amino acid sequence substantially shorter than the native sequence by means of one or more deletions.

The term “isolated” is used to indicate that the cell is detached from its parent organ and other organs, for example as neuronal and glial cells dissociated from brain tissue by physical or chemical dispersion such by techniques described in “The neuron in tissue culture” L. Haynes, ed., Wiley and Sons 1999; ISBN: 0471975052 or Protocols for Neural Cell Culture, 2^{nd} ed (Fedoroff S, and Richardson A., eds; otowa, N.J.: Humana Press).

The expression “neuronal dysfunctions as measured by electrophysiological detection” includes any changes in electrophysiological behavior of neuronal cells as compared to healthy wild-type neuronal cells or cells engineered to express a non-disease-causing protein; it includes for example a change in action potential characteristics such as changes in frequency, amplitude, shape, spike kinetics, number of spikes, spike/firing rate, number of population burst, waveform, epoch, temporal correlation between action potentials measured by any pair of electrodes, in particular a decrease in the number of population bursts, a decrease in the total number of spikes a change in spike or burst characteristics such as spike or burst kinetics or intensity.

### Engineered Neuronal Cells and Media for Cell Culture

Suitable genetically engineered neuronal cells for use in the present invention comprise electrically active primary neuronal cells which are genetically engineered to express a neurodegeneratively active protein. Examples include but are not limited to cortical or hippocampal neurons, for example from rat embryos, transduced with a lentiviral vector to express a neurodegeneratively active protein.

Genetically engineered neuronal cells for use in a method according to the invention may be produced by contacting neuronal cells with a self-inactivating HIV1-derived lentiviral vector (such as a SIN-W-PGK vector as described in Déglon et al., 2000 Human Gene Therapy, 11:179-190 and/or a SIN-W-TRE vector such as described in Régulier et al., 2004, Methods Mol. Biol. 2004; 277:199-213) encoding for at least one neurodegeneratively active protein, typically after about 0-7 days in culture.

Self-inactivating HIV1-derived lentiviral vectors suitable according to the invention comprise a tetracycline regulatory element (such as SIN-W-TRE, which contains seven direct repeats of the bacterial tetO tetracycline operator sequence upstream of a minimal Cytomegalovirus promoter) controlling the expression of an mRNA sequence encoding for the said at least one neurodegeneratively active protein gene combined with a self-inactivating HIV1-derived lentiviral vector encoding the tetracycline-controlled transactivator tTA1 (i.e. tetracycline repressor tetR fused to four copies (4F) of the minimal transcriptional activation domain of VP16) under the control of the mouse PGK (phosphoglycerate kinase) promoter. In a particular aspect, the self-inactivating HIV1-derived lentiviral vector is applied to the neuronal cell culture at a concentration of about 25 ng (e.g. 5-150 ng) p24 antigen/ml together with a lentiviral vector expressing the tetracycline-regulatable transactivator (tTA1) at a concentration of about 40 ng (e.g. 20-200 ng) p24 antigen/ml as described in Gambazzi et al., 2010, J. Phamacol. Exp. Ther., in press.

Neurodegeneratively active protein-encoding sequences to be inserted into these vectors include at least one sequence selected from sequences encoding mutant huntingtin protein carrying 82Q glutamines (SEQ ID NO: 1) or a fragment thereof (e.g. a fragment consisting of the first 171 amino acids of the mutant huntingtin protein carrying 82Q glutamines or a fragment consisting of the first 171 amino acids of the mutant huntingtin protein carrying 82Q glutamines coupled with linking sequences and myc tag and his tag sequences (SEQ ID NO: 2)), sequences encoding full-length amyloid precursor protein (APP) (SEQ ID NO: 3) or a variant thereof (e.g. APP with missense mutation KM/670/671/NL or missense mutation E693G or missense mutation V717F) or a fragment thereof, sequences encoding microtubule-associated protein tau (MAPT) (SEQ ID NO: 4) or a variant thereof (e.g. Missense MAPT mutation L618P) or a fragment thereof, sequences encoding full-length presenilin 1 (PSEN1) (SEQ ID NO: 5), or a variant thereof (e.g. PSEN1 missense mutation V82L or missense mutation V96F or deletions IM 83/84) or a fragment thereof, sequences encoding granulin (GRN) (SEQ ID NO: 6) or a variant thereof or a fragment thereof, sequences encoding alpha-synuclein (SNCA) isoform 1 (SEQ ID NO: 7), isoform 2-4 (SEQ ID NO: 8), isoform 2-5 (SEQ ID NO: 9) or a variant thereof or a fragment thereof, sequences encoding leucine-rich repeat kinase 2 (LRRK2) (SEQ ID NO: 10) or a variant thereof (e.g. missense mutation G2019S) or a fragment thereof, sequences encoding ATPase type 13A2 (ATP13A2) (SEQ ID NO: 11) or a variant thereof (e.g. missense mutation G504R) or a fragment thereof, sequences encoding superoxide dismutase 1 (SOD1) (SEQ ID NO: 12) or a variant thereof (e.g. missense mutation G93A) or a fragment thereof and sequences encoding dynactin 1 (DCTN1) (SEQ ID NO: 13) or a variant thereof (e.g. missense mutation M571T) or a fragment thereof.

In a particular embodiment, the neurodegeneratively active protein-encoding sequence to be inserted into self-inactivating HIV1-derived lentiviral vector contains a sequence encoding the first 171 amino acids of the huntingtin protein mutant fragment of SEQ ID NO: 2.

In a further particular embodiment, the neurodegeneratively active protein-encoding sequence to be inserted into self-inactivating HIV1-derived lentiviral vector encodes the first 171 amino acids of the huntingtin mutant protein carrying 82Q glutamines, wherein the nucleic acid sequence is represented by SEQ ID NO: 14.

In a particular embodiment, genetically engineered neuronal cells for use in a method according to the invention may be produced by contacting neuronal cells with a combination of several lentiviral vectors comprising encoding sequences for distinct neurodegeneratively active proteins or by contacting neuronal cells with a lentiviral vector comprising encoding sequences for several distinct neurodegeneratively active proteins. In a particular embodiment, the invention provides genetically engineered neuronal cells produced by contacting neuronal cells with a combination of a lentiviral vector encoding for full-length amyloid precursor protein (APP) (SEQ ID NO: 3) or a variant or fragment thereof, a lentiviral vector encoding for microtubule-associated protein tau (MAPT) (SEQ ID NO: 4) or a variant thereof or a fragment thereof, and a lentiviral vector encoding for full-length presenilin 1 (PSEN1) (SEQ ID NO: 5), or a variant thereof or a fragment thereof. In this case, neuronal cells can be contacted sequentially with several vectors or simultaneously, typically simultaneously. In another particular embodiment, the invention provides genetically engineered neuronal cells produced by contacting neuronal cells with a lentiviral vector encoding for full-length amyloid precursor protein (APP) (SEQ ID NO: 3) or a variant or fragment thereof, for microtubule-associated protein tau (MAPT) (SEQ ID NO: 4) or a variant thereof or a fragment thereof, and for full-length presenilin 1 (PSEN1) (SEQ ID NO: 5), or a variant thereof or a fragment thereof.

Suitable cell culture media includes nutritive material such as amino acids, vitamins, minerals, glucose, albumin, insulin, transferrin, triiodo-L-thyronine, L-carnitine, ethanolamine, galactose, putrescine, corticosterone, linoleic acid, lipoic acid, progesterone, retinols, and antioxidants such as vitamin E, catalase, superoxide dismutase, and glutathione, which are suitable for growth of a neuronal network or neurons Chen et al., 2008, J. Neurosci. Methods 171:239-247).

In a particular embodiment, the invention provides an isolated neuronal cell transduced by a lentiviral vector including a sequence encoding the huntingtin protein mutant fragment of SEQ ID NO: 2.

In a further embodiment, the invention provides an isolated neuronal cell transduced by a lentiviral vector comprising a nucleic acid sequence encoding the first 171 amino acids of the huntingtin mutant protein carrying 82Q glutamines and having a sequence represented by SEQ ID NO: 14.

In a further particular embodiment, the invention provides provides an isolated neuronal cell transduced by a lentiviral vector represented by SEQ ID NO: 16.

In another particular embodiment, the invention provides an isolated neuronal cell composition comprising a cell according to the invention. Typically, the cellular composition further comprises suitable cell culture media.

In particular, the invention provides a genetically engineered neuronal cell according to the invention of a use thereof for the electrophysiological detection of a neuroactive substance is a substance able to prevent, repress or treat a neuronal dysfunction characterizing Huntington's disease.

According to another embodiment, the invention provides a kit for electrophysiological detection of a neuroactive substance, the kit comprising genetically engineered neuronal cells or a composition thereof or neuronal cells together with vectors to genetically engineer neuronal cells, wherein the genetically engineered neuronal cells or the neuronal cells once transduced with the said vectors express a neurodegeneratively active protein.

According to a further embodiment, the invention provides a kit for electrophysiological detection of a neuroactive substance, wherein the kit comprises genetically engineered neuronal cell according to the invention or a composition thereof or neuronal cells together with lentiviral vectors according to the invention to genetically engineer said neuronal cells.

Typically, a kit according to the invention further comprises instructions for use. In a particular aspect, a kit according to the invention further comprises a multielectrode array (MEA) optionally pre-coated with at least one surface modifying agent suitable for neuronal cell culture or optionally provided together with at least one surface modifying agent suitable for neuronal cell culture for coating the said MEA (e.g. laminin or poly-D-lysine). Typically, the MEA is stored under sterile conditions within the kit.

The culture medium may further comprise a candidate substance of interest or a composition thereof, the effect of the addition of which to the cell culture or of the removal thereof from the cell culture is monitored by the method according to the invention.

### Electrophysiological Activity Measurements & Microelectrode Array

According to one aspect is provided an ex-vivo method for the detection of a neuroactive substance as described herein.

According to a further aspect, an ex-vivo method for the detection of a neuroactive substance is provided wherein the neurodegeneratively active protein is a mutant of huntingtin protein (SEQ ID NO: 1) or a fragment thereof. Typically, in this case the neuroactive substance is a substance able to prevent, repress or treat a neuronal dysfunction characterizing Huntington's disease or condition.

According to another further aspect, an ex-vivo method for the detection of a neuroactive substance is provided according to the invention, wherein the genetically engineered neuronal cells are produced by a method comprising a step of contacting a neuronal cell with a self-inactivating HIV1-derived lentiviral vector comprising a nucleic acid sequence encoding said neurodegeneratively active protein operably linked to at least one sequence which controls expression of the corresponding protein.

According to another further aspect, an ex-vivo method for the detection of a neuroactive substance is provided according to the invention, wherein the neurodegeneratively active protein is a mutant huntingtin fragment of SEQ ID NO: 2.

According to another further aspect, an ex-vivo method for the detection of a neuroactive substance is provided according to the invention, wherein the genetically engineered neuronal cells are produced by a method comprising a step of contacting a neuronal cell with a lentiviral vector comprising a nucleic nucleic acid encoding sequence for the first 171 amino acids of the huntingtin protein carrying 82Q glutamines, wherein the nucleic acidsequence is represented by SEQ ID NO: 14.

According to another further aspect, an ex-vivo method for the detection of a neuroactive substance is provided according to the invention, wherein the genetically engineered neuronal cells are produced by a method comprising a step of contacting a neuronal cell with a self-inactivating HIV1-derived lentiviral vector of SEQ ID NO: 16.

According to another further aspect, an ex-vivo method for the detection of a neuroactive substance is provided according to the invention, wherein the genetically engineered neuronal cells are genetically engineered neuronal cells according to the invention.

According to another further aspect, an ex-vivo method for the detection of a neuroactive substance is provided according to the invention, wherein the electrophysiological response parameter is measured by a MEA.

Electrophysiological activity of the neuronal cells (e.g. spontaneous electrical activity) can be measured by the extracellular voltage for example at a point where the electrode tip of a MEA contact the neuronal cell sample. This measurement can be performed under sterile conditions (for example by placing in a presterilized closed incubator or container or covering with a semipermeable membrane or as described in Potter and De Manse, 2001, J. Neurosci. Methods, 110(1-2):17-24). In particular, this could be achieved by plating the neuronal cells on a multielectrode array (MEA) such as described in Marom and Shahaf, 2002, Quart. Rev. Biophys., 35: 63-87 and WO 2008/004010 and recording the electrical pulses spontaneously generated and propagated by neurons. These pulses can be measured by an electronic amplifier and a computer-based recording system, comprising a computer data acquisition system such as those commercialized by Multichannel System, GmBH (Reutlingen, Germany) and by Ayanda Biosystems SA (Lausanne, Switzerland). Typically, recorded signals are raw extracellular voltage deflections, amplified about 100 times from each electrode independently. Data acquisition software (such as MCRack, by Multichannel Systems GmBH, Reutlingen, Germany) settings are adjusted with appropriate software/hardware configurations, selecting the number and layout of the electrodes to be recorded.

According to another further aspect, an ex-vivo method is provided for the detection of a neuroactive substance according to the invention, wherein step (iii) comprises a data processing step that can be implemented by a computer to analyze changes in at least one action potential characteristics of the cells upon exposure to the candidate substance or candidate substance composition.

Neuronal activity being dependent on environmental conditions including temperature and pH, electrophysiological activity recording typically takes place in an incubator compatible with the electronic hardware of the apparatus for recording electrophysiological activity. Advantageously, the recording apparatus presents in its part which is in direct contact with the MEA, a temperature controlling element allowing to control and regulate the temperature of this part at about 1-2° C. lower than the temperature of the incubator in which the measurements are performed to prevent the formation of water condensation from the inside part of the cover that seals individual wells. Typically, the bottom part of the recording apparatus which is in direct contact with the MEA sits on a temperature regulated copper plate.

In a particular arrangement, a MEA containing neuronal cells on its inner surface for use in a method according to the invention may be stored in a Petri dish to protect it from damage or contamination, in a temperature regulated storage incubator. The MEA is then removed from the Petri dish and placed in the apparatus for recording electrophysiological activity, matching the position of the internal ground electrode. Recording can start as soon as the MEA is connected in the recording apparatus.

In a particular embodiment, the MEA comprises at least 59 electrodes and one internal ground electrode. According to a particular aspect, for a better accuracy of the results and, in particular, for measuring the correlation between the time of occurrence of spikes at different MEA electrodes, the number of channels are 59 or more.

In a particular embodiment, the neuronal cells are plated on a MEA after coating the MEA surface with a surface modifying agent such as polyethylenimine (e.g. 10 mg/ml) and laminin (e.g. 0.02 mg/ml) in Neurobasal medium or poly-L-lysine (0.01%) in water, which allows the attachment of cells and the formation and extension of networks of neurites.

Observables and specific read-outs characterizing neuronal (dys)function and its restoration that could be measured in a method according to the invention are standard parameters employed in cellular electrophysiology. In particular, electrophysiological activity recording includes recording of spontaneous action potential and culture-wide bursting profiles. For example, electrophysiological activity comprises neuronal firing rates measured by the instantaneous number of spikes detected per electrode and per unit of time. Each spike is typically identified by detecting the time when the raw voltage signals exceeds an amplitude threshold, set arbitrarily as 6 times the level of the noise, as described in Wagenaar D, PhD Thesis, California Institute of Technology, 2006, number and frequency of occurrence of epochs of synchronous activity (i.e. defined as the simultaneous occurrence of detected peaks across several MEAs electrodes, within a time window of less than 50 msec), temporal correlations (i.e. computed by the cross-correlation measure) across all the possible pairs of electrodes (e.g. 59 electrodes, 59*58 pairs), by counting the number of times two electrodes detected synchronous spikes.

### Analysis of Electrophysiological Measurements

Recorded extracellular voltage signals are analyzed off-line by a computer data analysis system, to analyze for example the amplitude of the spikes detected independently at each MEA electrode or their overall number within some recording time. Typically, a temporal analysis of the action potential profiles or the changes thereof is carried out. The analysis of network-level spiking activity may be carried out by mathematical and computer models of the electrical activity of neurons and networks such as described in Giugliano et al., 2004, J. Neurophysiol., 92(2):977-96.

According to a further aspect, a method of the invention is provided wherein the measurement of the said at least one electrophysiological response includes measuring the neuronal network's ability to respond to an external electrical stimulus. Typically, the measurement of a neuronal physiological parameter such as the ability to respond to an external electric stimuli is measured similarly as for spontaneous activity and includes the timing and magnitude of responses relative to the position of the stimulating electrode, for example by counting how many spikes are detected in the 200 millisecond following the electric stimuli, and subtracting this number by the average number of spikes detected when no stimulation is applied.

According to a further aspect, a method of the invention is provided wherein the measurement of the said at least one electrophysiological response is coupled with the recording of at least one morphological and/or structural parameter of the neuronal cells, in particular morphological details of the neurons, inter-neuronal connectivity patterns (e.g. size of the cell body, number, shape, and length of the neurites protruding from the soma, shape and number of synaptic boutons, etc.). Typically, the measurement of morphological and/or structural parameters of the neuronal cells may be carried out by microscopy at a plurality of regions in said neuronal cell culture sample (e.g. on the MEA), followed by an image analysis and compared to at least one morphological and/or structural parameter of the neuronal cells in the absence of candidate substance or candidate substance composition.

In particular, in a particular embodiment, a method according to the invention comprises the following further steps:

(iia) Comparing at least one morphological and/or structural and/or physiological parameter of the neuronal cells measured simultaneously at a plurality of regions in said neuronal cell culture sample when contacted with a candidate substance or a candidate substance composition with at least one baseline for the said morphological and/or structural parameter of said regions;
(iib) Determining the difference between said morphological and/or structural parameter and/or physiological and said baseline morphological and/or structural parameter and/or physiological;
(iic) Detecting the presence or absence of a neuroactive substance in said candidate substance or candidate composition based upon the difference determined under step (iib) or detecting a neuronal adverse effect of said candidate substance or candidate substance composition based upon the difference determined under step (iib); wherein steps (iia), (iib) and (iic) may be performed either sequentially with steps (ii), (iii) and (iv) or in parallel.

According to one further aspect of the invention, is provided a method according to the invention wherein step (iii) comprises a data processing step that can be implemented by a computer to analyze changes in at least one action potential characteristics of the cells upon exposure to the candidate substance or candidate substance composition.

According to another further aspect of the invention, is provided a method according to the invention wherein step (iic) comprises a data processing step that can be implemented by a computer to analyze changes in at least one morphological and/or structural parameter and/or physiological of the cells upon exposure to the candidate substance or candidate substance composition.

According to a further aspect of the invention, a method according to the invention is provided wherein the said at least one changes in the action potential characteristics is selected from changes in frequency, amplitude, shape, spike kinetics, number of spikes, spike/firing rate, number of population burst, waveform, epoch, temporal correlation between action potentials measured by any pair of electrodes.

According to another further aspect of the invention, a method according to the invention is provided wherein the said at least one changes in the action potential characteristics is derived from spike-time histograms (STH), which graphically displays at any moment the number of spikes detected from all MEA electrodes.

A method according to the present invention has the major advantages to monitor electrophysiological activity in intact post-mitotic neurons spontaneously firing action potentials thereby enabling screening for neuroactive substances devoid of undesired neuronal side effects. Further, a method according to the invention allows the monitoring of electrophysiological activity, in particular of early electrophysiological dysfunctions, over a days-to-weeks timescale in highly relevant neuronal cellular model systems which more closely and realistically emulates the chronic processes actually leading to human neurodegeneration and neurotoxicity. Additionally, a method according to the invention also allows detection of potential neuronal adverse effects, e.g. toxicity, related to the presence of a neuroactive substance, thereby providing a means to detect neuroactive effects that are detrimental as well as the ones that are positive (a single substance can show one or the other or both [positive and/or negative] effects) for assessing the balances of therapeutic effects and “side effects” of therapeutic candidate substances.

Since it is impossible to obtain neurons from people suffering from neurological disorders (biopsies) in sufficient quantities for research, the present invention combines known human disease-causing agents (genes) with neural cells by lentiviral transduction ex vivo.

The invention having been described, the following examples are presented by way of illustration, and not limitation.

### EXAMPLES

The following abbreviations refer respectively to the definitions below:

dB (decibel), dec (decade), Gb (Gigabyte), F (Farad), h (hour), Hz (herz), min (minute), M (molar), U (Ohm), s (second), BNDF (brain-derived neurotrophic factor), cDNA (complementary DNA), DIV (Days In Vitro), DNA (Deoxyribonucleic acid), HD (Huntington's disease), htt (huntingtin), IBI (the inter-burst time interval), MEA (multielectrode array), PB (population burst), PBS (Phosphate buffer saline), PCR (Polymerase Chain Reaction), qPCR (quantitative PCR), R.H. (Relative Humidity), RNA (Ribonucleic acid), S.E.M. (standard error of the mean).

### Example 1

### Method According to the Invention Using Huntington's Disease Cell Model

Neuronal dysfunction in Huntington's disease (HD) is accompanied by specific clinical manifestations involving motor and cognitive impairments, including chorea, depression, and/or difficulties in decision-making. At the cellular level, HD appears to arise from neurotoxicity involving a state of cellular dysfunction preceding cell death. During this process, intracellular exposure to mutant huntingtin (htt) and N-terminal htt fragments containing the expanded polyglutamine domain lead to protein aggregation, abnormalities in cellular signaling and trafficking, and the dysregulation of gene expression (Luthi-Carter et al., 2007, Drug Discovery Today: Disease Mechanisms, 4: 111-119).

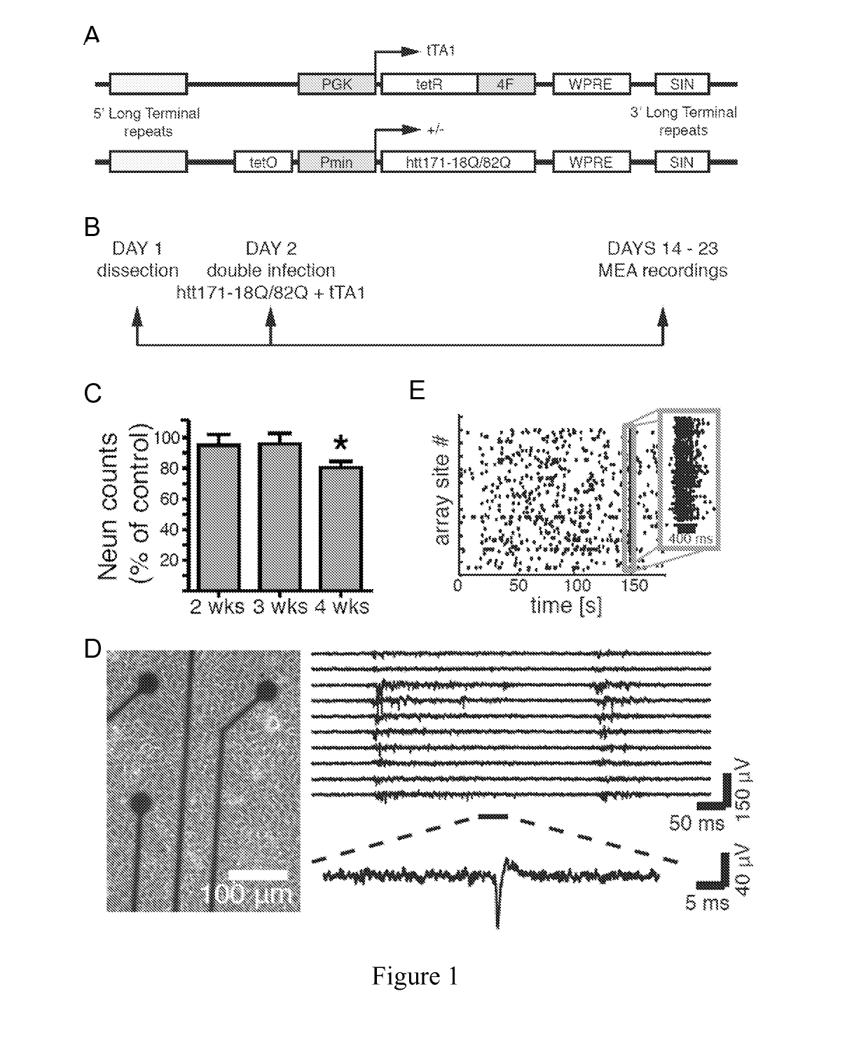

Cells modeling in-vitro HD-related cortical dysfunction were engineered for use in a method according to the invention for showing that the effects of molecular changes on synaptic changes in cortical microcircuits exposed to mutant htt fragments could be measured by a method according to the invention. To this end, cortical cells E16-E19 Wistar rat embryos (Charles River, France) were engineered by lentiviral gene transfer using Human Immunodeficiency Virus Type 1 (HIV-1)-derived vectors to express the first 171 amino acids of normal or mutated htt under the control of a tetracycline response element-containing promoter as described in Rudinskiy et al., 2009, J. Neurochem., 111: 460-472 (FIGS. 1A,B) to achieve high transduction efficiency and high levels of transgene expression in rat primary cortical neurons following the protocol below.

### Lentiviral Vector Production and Infection

Plasmids encoding the first 171 amino acids of mutated htt (htt171-82Q) (FIG. 6, SEQ ID NO: 14) or wild-type htt (htt171-18Q) (FIG. 6, SEQ ID NO: 15) under the control of a tetracycline-regulated element promoter, or the tetracycline-regulatable transactivator (tTA1) under the control of a phosphoglycerate kinase promoter were used to prepare self-inactivating lentiviral vectors (FIG. 1A) as described previously (Regulier et al., 2003, Hum. Mol. Genet., 12: 2827-2836). Lentiviral particles were re-suspended in phosphate-buffered saline (PBS)+1% bovine serum albumin and the particle content of viral batches was assessed by p24 ELISA (RETROtek, Gentaur, Paris, France) (Viral titering involves capture of p24 viral protein from the sample on a microtiter plate and detection with a second anti-p24 antibody and visualization via a colorimeteric horseradish-generated product detected at 450 nm) Cells were plated as described below. On the day following cell plating, cultures were infected with lentiviruses at ratios of 150 ng p24/1000 cells (tTA1) and 120 ng p24/1000 cells (htt171-82Q or htt171-18Q) (FIG. 1B).

### Cell Cultures

Cortical cells were prepared and cultured as described previously (Van Pelt et al., 2004, IEEE Trans. Biomed. Eng., 51: 2051-2062). Cells were plated on multielectrode arrays (MEAs) and/or culture dishes, with prior surface coating by polyethylenimine (10 mg/ml, Fluka) and laminin (0.02 mg/ml, Gibco) in Neurobasal medium (MEAs) or poly-L-lysine (0.01%) in water (culture dishes), respectively, which allow the cells to readily attach to the surface and extend neurites. Plating density was 1′500 cells/mm² for MEAs, allowing for quick network formation and high multichannel electrophysiological recordings efficiency. A lower density of 500 cells/mm² was used for culture dishes, to facilitate cell counting and morphological analysis. Medium containing Neurobasal, 2% B-27 supplement (GIBCO, Invitrogen Corporation, ref 17504) and 10% horse serum (from GIBCO, Invitrogen Corporation, ref. 16050-130) was changed three times per week by removing 0.7 ml and adding 1 ml of fresh medium. Where indicated, recombinant human BDNF (R&D Systems, Minneapolis, Minn., USA) at a concentration of 50 ng/ml was added to the medium twice a week starting from in vitro day 4.

Immunodetection with an anti-polyglutamine antibody (2B4) as described below showed that cultures expressing htt171-82Q accumulated the transprotein (htt171-82Q) in the nuclear compartment and developed neuritic htt within 10-14 days after lentiviral transduction, whereas cultures expressing htt171-18Q exhibited only weak and diffuse, primarily non-nuclear, labeling. As assessed by the numbers of NeuN-positive nuclei counted as described below, the neuronal viability in htt171-82Q-expressing cortical cultures showed no diminution compared to htt171-18Q-infected controls up to 3 weeks in vitro, but showed a progressive loss of neuronal cells at later timepoints (≧3.5 weeks) (FIG. 1C).

### Immunostaining and Image Analysis

Cultures were washed with PBS and fixed in 4% paraformaldehyde (Fluka/Sigma, Buchs, Switzerland) or in methanol (Merck, Germany) for PSD95 (Post-synaptic density protein of 95 kDa) staining for 10 min at 4° C. Cultures were incubated with NeuN antibody which reacts with fox-3 (1:500, Chemicon, Temecula, Calif.) or 2B4 antibody which reacts with huntingtin (1:500 Millipore, Zug, Switzerland), antibody which reacts with postsynaptic density protein of 95 kDa (PSD95) (Sans et al., 2000, J. Neurosci. 20:1260-71) (1:200, Affinity Bioreagents, Golden, Colo.) and antibody which reacts neuronal class III beta-tubulin Tuj1 (Jepsen et al., 2000, Cell 102(6): 753-763) (1:2000, Covance, Emerville, Calif.) in PBS containing 10% normal goat serum (NGS, Gibco, Invitrogen, Basel, Switzerland) and 0.1% Triton X-100 (TX, Fluka, Sigma, Buchs, Switzerland). Cultures were rinsed three times in PBS and then incubated for 1 hour with fluorescent goat anti-mouse secondary antibodies (for NeuN: 1:1000 Cy3-conjugated antibody from Jackson Immunoresearch Laboratories, WestGrove, Pa.; for 2B4: 1:1000 Alexa Fluor® 488-conjugated antibodies from Invitrogen, Basel, Switzerland, for Tuj1: 1:1000 Alexa Fluor® 594-conjugated antibody from Invitorgen, Basel, Switzerland). Where indicated, cultures were stained with Hoechst 33342 dye (Invitrogen, Basel, Switzerland). Images of NeuN-labeled cultures (n=5 or 6 per condition) were acquired using BD Pathway 855 (BD Biosciences, San Diego, USA) microscope under non-saturating exposure conditions, using the same acquisition settings for all samples in a given experiment. Images of PSD95, 2B4 and Tuj3 stained cultures were acquired using laser-scanning confocal microscope (TCS-SP2 AOBS, Leica Microsystems, Wetzlar, Germany), using the same acquisition settings for all samples in a given experiment unless specifically noted otherwise. Counting of NeuN-positive nuclei and PSD95-positive plasma membrane subdomains (punctae) was performed with ImageJ software (NIH, Bethesda, Md., USA) applying intensity and size thresholds.

The engineered cells were used in a method according to the invention where their spontaneous electrical activity (e.g. the number of electrical discharges (spikes), each directly related to nerve impulses, detected in a time interval by each MEA electrode—firing rate) was recorded by an extracellular multielectrode array (MEA) device as described below. The effect of mutant htt fragment expression on cortical neuron network activity was thereby monitored.

### Multielectrode Arrays (MEAs)

Commercial MEAs with planar TiN substrate electrodes (MultichannelSystems, Reutlingen, Germany) such as described in Potter and De Marse, 2001, above were employed for recording over long-term experiments the spontaneous electrical activity of mammalian neurons, maintained under healthy conditions ex-vivo. Substrate electrodes had a diameter of 30 μm (FIG. 1D), and were organized in an 8×8 square grid with 200 μm spacing. Cell maintenance and electrophysiological recordings were performed at 37° C., 9% O₂, 5% CO₂, and 65% R.H. (Potter and DeMarse, 2001, above), inside a low-humidity incubator comprising an electronic-friendly environment (Jouan IG750, ThermoFischer Scientific, Waltham, Mass., USA). MEA electrodes had an impedance of 100 kΩ (in PBS). Electronic amplifiers (MultichannelSystems, Reutlingen, Germany) had a standard gain of 61.6 dB, between 10 Hz and 3 kHz (filters +−60 dB/dec), and large input-impedance (10¹¹Ω in parallel to 10 pF). Recordings were performed at least 24 h after medium changes except as indicated (for experiments with the trkB inhibitor K252a).

### MEA Data Acquisition and Analysis

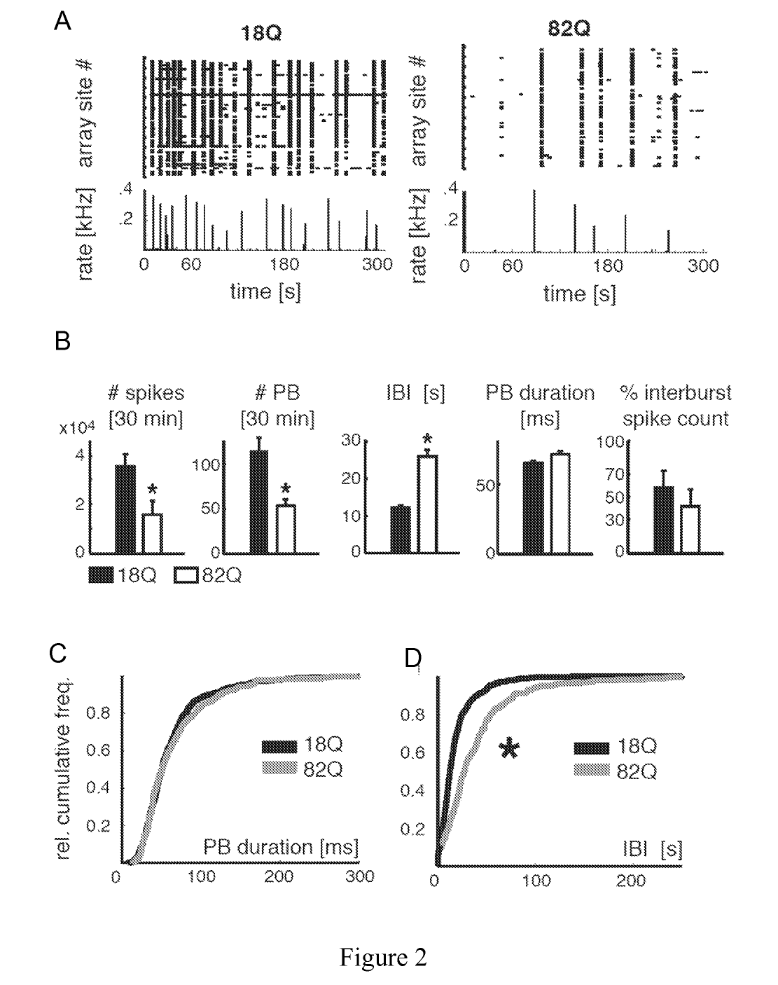

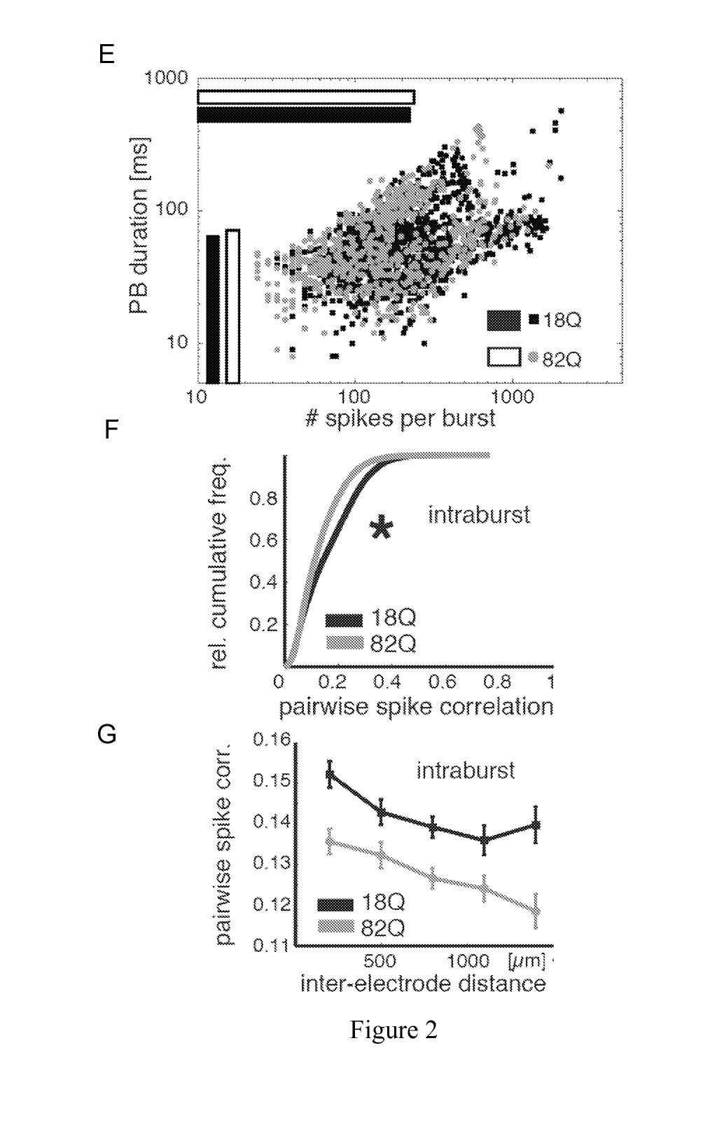

After 5 min of accommodation time following the mounting of each MEA inside the recording setup, spontaneous electrical activity from the cultured neurons was detected, amplified, and recorded at each of the 60 substrate extracellular electrodes simultaneously for 30 min at 20 kHz/channel. Recorded traces consisted in electric voltage signals whose sudden small large amplitude fluctuations are known to be produced by the nerve impulse discharges emitted by neurons growing in close proximity to the substrate electrodes that detect them extracellularly, directly related to the firing of action potentials by the neurons in the proximity of each electrode. MCRack software (MultichannelSystems, Reutlingen, Germany) was used to acquire and store the data (9 Gb/session, split into ˜2 Gb files), which were processed off-line channel by channel. Raw voltage waveforms (FIG. 1D, bottom right) were digitally filtered between 150 Hz and 2.5 kHz and fully rectified. The occurrence of an action potential at a given electrode was identified by a peak-detection algorithm, based on the crossing of an adaptive threshold, as in the LimAda algorithm (Wagenaar 2006, above). Recorded events included the number of milliseconds since the start of the experiments, specifying the time of occurrence of each spike, the index of the electrode where it was detected, and the preceding 200 ms and following 800 ms of the corresponding raw voltage trace (FIG. 1D—right panels and FIG. 1E). The occurrence and duration of the time interval characterized by synchronized firing (i.e. bursts of spikes; FIGS. 1D, top right and E) were identified and detected as in van Pelt et al., 2004, above, by post-processing the spike-time histograms (STH). This method of extracellular recording and analysis enabled to distinguish between epochs of asynchronous spiking activity and epochs of population-wide bursting. During each recording session, the overall number of bursts, the statistics of the Inter-Burst time Intervals (i.e. abbreviated with I.B.I.s), and the number of individual spike that composed each burst were employed as observables of the network-level emerging spontaneous activity of the cultured neurons (FIGS. 2A-E, 4A-D). Data are expressed as the mean±S.E.M. Student's t-test and the Kolmogorov-Smirnov test were used to assess the significance (p<0.05) of differences in averages and cumulatives, respectively. Pair-wise correlations between intra-burst spike-trains were evaluated as the peak amplitude of the spike-triggered cross-correlograms over a fixed time window (varied in the range 1 ms-1 s) (Gruen, 2009, J. Neurophysiol., vol. 101 (3) pp. 1126-1140). After each recording session, single-unit activity was detected at each electrode by an adaptive threshold algorithm as described above. Occurrence of individual spike times for each MEA electrode was then represented as a raster diagram (FIG. 1E), where dots are plotted in vertical correspondence to the electrode, the spike has been detected from and in horizontal correspondence to the time of occurrence, and summarized as spike-time histograms (STHs) which counts for all the electrodes available how many dots have been drawn for the same horizontal coordinate (FIG. 2A). These histograms estimate the instantaneous firing rate across all recording sites binned in constant time intervals (≦1 s). PBs were then identified as increased synchronous firing epochs and detected as peaks in the STH (Van Pelt et al., 2004, above) (FIG. 2A). The PB duration was conventionally defined and estimated as the interval during which the instantaneous firing rate persisted above 5% of its local peak amplitude, computed by means of the raster plot for better temporal accuracy.

### Mathematical Model of a Cultured Neuron Network

A simplified spike-rate model accounting for the patterned electrical activity emerging in populations of cultured neurons was defined and computer-simulated to interpret the electrophysiological recordings. Firing rate R(t) of the ensemble of cortical neurons in terms of the single-cell f-I curve and of recurrent connectivity were described in Giugliano et al., 2008, Biol. Cybern., 99: 303-318 and La Camera et al., 2008, Biol. Cybern., 99: 279-301). The model replicates some of the features characterizing the population bursts as transient irregular outbursts in R(t). Full details and the source code are provided as a ModelDB entry (https://senselab.med.yale.edu/modeldb/ShowModel.asp?model=125748) (Hines et al., 2004, J. Comput. Neurosci., 17, 7-11).

Whereas htt171-82Q- and htt171-18Q-expressing cells were indistinguishable at early timepoints (≦2 weeks), disturbances in collective neuronal spiking activity in htt171-82Q-expressing cells started to be observed between 2 and 3 weeks in vitro (FIG. 2). The fact that the collective firing behavior in htt171-82Q- and htt171-18Q-expressing cultures was similar initially suggests that the htt171-82Q protein does not have a major effect on ex vivo neuronal development. Direct inspection of raw MEA voltage recordings revealed a prominent temporal organization of spontaneous electrical activity in the form of bursts of spikes (i.e. time interval with a large number of spikes) in htt171-82Q- and htt171-18Q-expressing cells (and also in uninfected cells). The mean spike and population burst (PB) numbers and the distribution of the inter-burst-intervals (IBIs) differed significantly between htt171-18Q and htt171-82Q cultures (FIG. 2A-B), with spikes and PBs being significantly less frequent in htt17′-82Q neuronal networks (FIG. 2B,D). In contrast, the average fraction of asynchronous (i.e. inter-burst) firing showed no difference between the two conditions (FIG. 2B). These observations suggest a specific impairment (or impairments) of network communication underlying PB ignition, and not a change in cell excitability in the present HD model. This stereotypical character of PBs was further confirmed in both the disease (htt171-82Q) and control conditions by comparing the cumulative distributions of PB duration and of the inter-burst-interval (FIG. 2C,D). In addition, the overall number of action potentials participating in each PB (i.e. the intra-burst firing) revealed no significant difference between htt171-82Q- and htt171-18Q-expressing cells.

As htt171-82Q expression was neither accompanied by cell loss nor by decreased intra-burst firing, the observed electrophysiological dysfunctions could not be attributed as the result of decreased spiking excitability nor of reduced electrode spike transduction. This was further supported by the observation that both synchronous/bursting and asynchronous modes of firing demonstrated similar features in the mutant and wild-type cultures (FIG. 2). Computer-simulating mathematical model of an ex vivo cortical network as described above shows that irregular PBs emerge as spontaneous transitions between a “resting” (i.e. asynchronous firing) state and an “excited”, transiently self-sustaining (i.e. synchronous firing) state (Giugliano et al., 2008, above). Although impairments in intrinsic excitability, such as the spike-frequency adaptation mediated by calcium-dependent outward currents, would interfere with PB by affecting its duration, the model predicts that the frequency (not the duration) of spontaneous PB occurrence depends on the recurrent excitatory connectivity and on the efficacy of the synaptic coupling between neurons.

Pair-wise correlations among spike trains (e.g. sequences of subsequent action potentials, detected at the same electrode) were also evaluated as an indirect measure of the degree of functional connectivity in the network (Perkel et al., 1967, Biophysical Journal, 7, 419-440). Assessed across all possible MEA electrode pairs, weak correlations were found between the firing activity in both in htt171-82Q and htt171-18Q cultures. However, pair-wise correlations were significantly smaller in the HD model than in the control, while no qualitative difference was observed when the (decreasing) relationship between correlation and inter-electrode pair distance was evaluated (FIG. 2G). This has been studied and shown in FIG. 2F-G, where the numerical values representing the similarity of the times of occurrence of spikes (i.e. the cross-correlation), detected at two generic electrodes of the same MEA has been counted and displayed as a cumulative distribution plot. Because the gray line is displaced towards the left (FIG. 2F), then the cross-correlations were globally smaller for the disease culture.

Overall, the above modelling and recording results suggested that a discrete subset of electrophysiologic measures differentiate htt171-82 Q- and htt171-18 Q-expressing cells. Specifically, the rate of PB occurrence and the spike-train correlations are significantly decreased in htt171-82Q-compared to htt171-18Q-expressing cells, consistent with a deficit in synaptic connectivity rather than a decrease in intrinsic excitability. This difference is also accompanied by a lower number of total spikes in the htt171-82Q-expressing cultures. In contrast, the spontaneous emergence of PBs (stereotypical of recurrent glutamatergic synaptic interactions in random networks as described in Marom and Shahaf 2002, Quart. Rev. Biophys., 35: 63-87 and PB spreading to the entire network demonstrated similar characteristics in htt171-82Q and htt171-18Q cells. Likewise, the synaptic and/or cellular dynamics underlying PB termination (generally termed activity-dependent fatigue by intracellular ion accumulation or neurotransmitter ready-releasable pool exhaustion), was also similar in htt171-82Q and htt171-18Q cultures (as evidenced by the number of spikes/PB and the PB duration remaining unaffected).

The cause of the observed decrease in PB firing behaviour was investigated. An inhibitor of TrkB receptor (inhibiting BDNF signalling through inhibition of TrkB receptors), K252a, was shortly applied to the cell culture as described below.

### TrkB Inhibitor Experiments

Experiments were initiated on cultures prepared on MEAs (n=9) as described above starting at 14 DIV. Spontaneous activity was recorded for 30 min in three sessions. The first comprised recordings of all cultures 5 hours prior to initiating pharmacologic treatments. The second comprised recordings of all cultures 45 minutes after treatments with TrkB inhibitor K252a (Sigma cat. K1639) at a concentration of 10 nM to 3 cultures, at 100 nM to 3 separate cultures, and maintaining 3 untreated cultures as controls. A third session recorded the behaviour of all three groups of cells one day later (at 15 DIV). Analyses of MEA recordings were performed as indicated above.

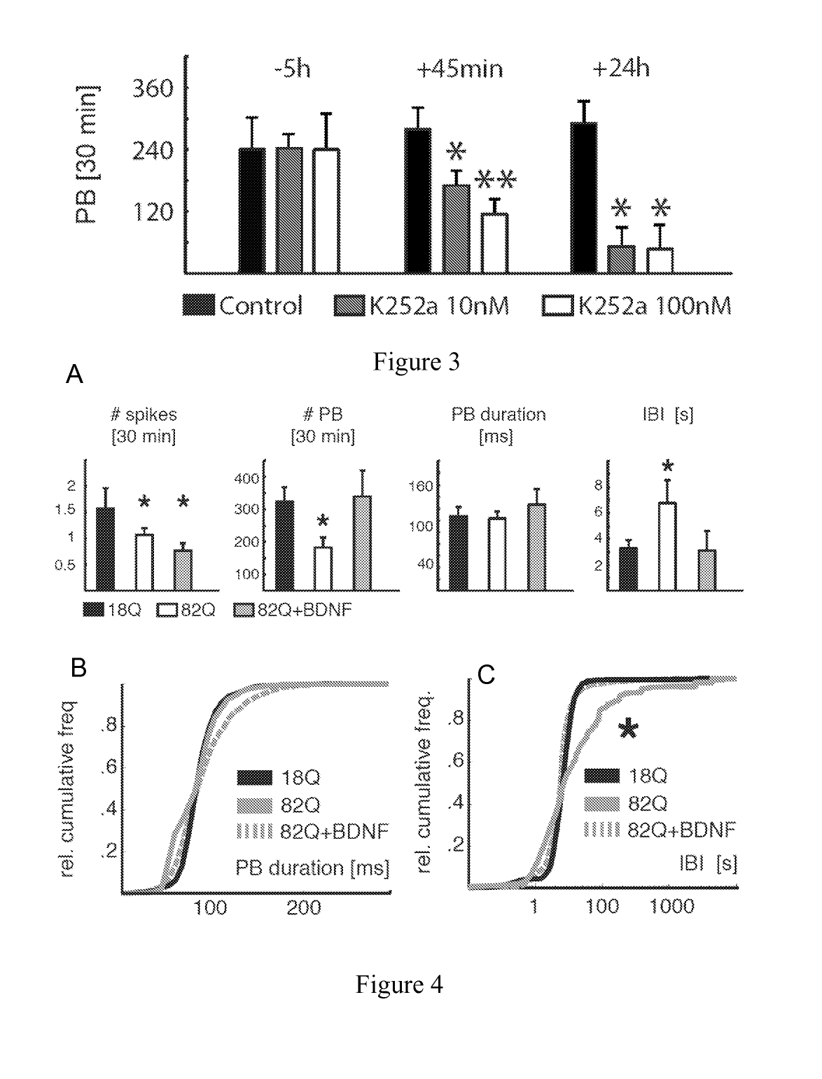

Treatments with the TrkB inhibitor resulted in a dose- and time-dependent loss of PB firing (FIG. 3), without influencing neuronal viability (as determined by NeuN counts), suggesting that decreased brain-derived neurotrophic factor (BDNF) activity (and potentially decreased BDNF expression) might underlie the observed diminution of function observed in htt171-82Q cells.

BDNF expression was then measured in both resting and depolarizing conditions at timepoints preceding or coincident with altered network behaviour as described below.

### BDNF RNA Measurements

Neuronal stimulation was performed starting 1.5 weeks after infection with lentivirus. In order to reduce endogenous synaptic activity and prevent calcium entry through N-methyl d-aspartate (NMDA) receptors, cortical neurons were pretreated for 30 min with 1 μM tetrodotoxin (Alexis), 100 μM d-(−)-2-amino-5-phosphonopentanoic acid, D(−)-AP-5 (Sigma), and 40 μM 6-cyano-7-nitroquinoxaline-2,3-dione, CNQX (Sigma). To increase the specificity of the stimulations, neurons not subjected to stimulation were exposed to 10 μM nifedipine (Sigma) and 20 μM N-[2-(p-Bromocinnamylamino)ethyl]-5-isoquinoline sulfonamide.2HCl, H-89 (Calbiochem) (in order to inhibit endogenous neurotransmitter activity). Where indicated, neurons were stimulated for 90 min with 10 μM forskolin (Calbiochem) and 30 mM KCl. Total RNA was extracted using the RNeasy kit (Qiagen) (using silica membrane columns) and 800 ng of total RNA were used for cDNA synthesis employing High Capacity RNA to cDNA RT kit (Applied Biosystems) (using MuLV polymerase and RNase inhibitor protein). Quantitative real-time PCR was performed on a 7900HT Real-Time PCR System with SDS 2.3 software (Applied Biosystems) using Power SYBR green PCR master mix (Applied Biosystems). Relative expression was calculated by normalization of BDNF to B-actin expression as described in Zucker et al., 2005, Hum. Mol. Genet., 14: 179-189. All qPCR reactions were performed in triplicate. Primers used in this study were based on previous assays (Chen et al., 2003, Science, 302: 885-889; Kobayashi et al., 2008, Brain Res., 1206: 13-19) and are listed in Table 1 below.

TABLE 1

| Exon | Forward primer | Reverse primer |
|---|---|---|
| I | tgttggggagacgagatttt | cgtggacgtttgcttctttc |
| | (SEQ ID NO: 17) | (SEQ ID NO: 18) |
| IIa | tacttcatccagttccaccag | caagttgccttgtccgt |
| | (SEQ ID NO: 19) | (SEQ ID NO: 20) |
| IIb | aagctccggttccaccag | tgcttctttcatgggcg |
| | (SEQ ID NO: 21) | (SEQ ID NO: 22) |
| IIc | gtggtgtaagccgcaaaga | ccgtggacgtttgcttctttc |
| | (SEQ ID NO: 23) | (SEQ ID NO: 24) |
| III | ctgagactgcgctccactc | gtggacgtttgcttctttca |
| | (SEQ ID NO: 25) | (SEQ ID NO: 26) |
| VI | gatccgagagctttgtgtgg | gtggacgtttgcttctttca |
| | (SEQ ID NO: 27) | (SEQ ID NO: 28) |
| IV | cgccatgcaatttccactatcaataatttaac | gtttactttgacaagtagtgactgaaaaag |
| | (SEQ ID NO: 29) | (SEQ ID NO: 30) |
| Universal | tggatgccgcaaaca | ccgggactttctccaggact |
| | (SEQ ID NO: 31) | (SEQ ID NO: 32) |
| β-actin | aggcatcctgaccctgaag | gctcattgtagaaagtgtgg |
| | (SEQ ID NO: 33) | (SEQ ID NO: 25) |

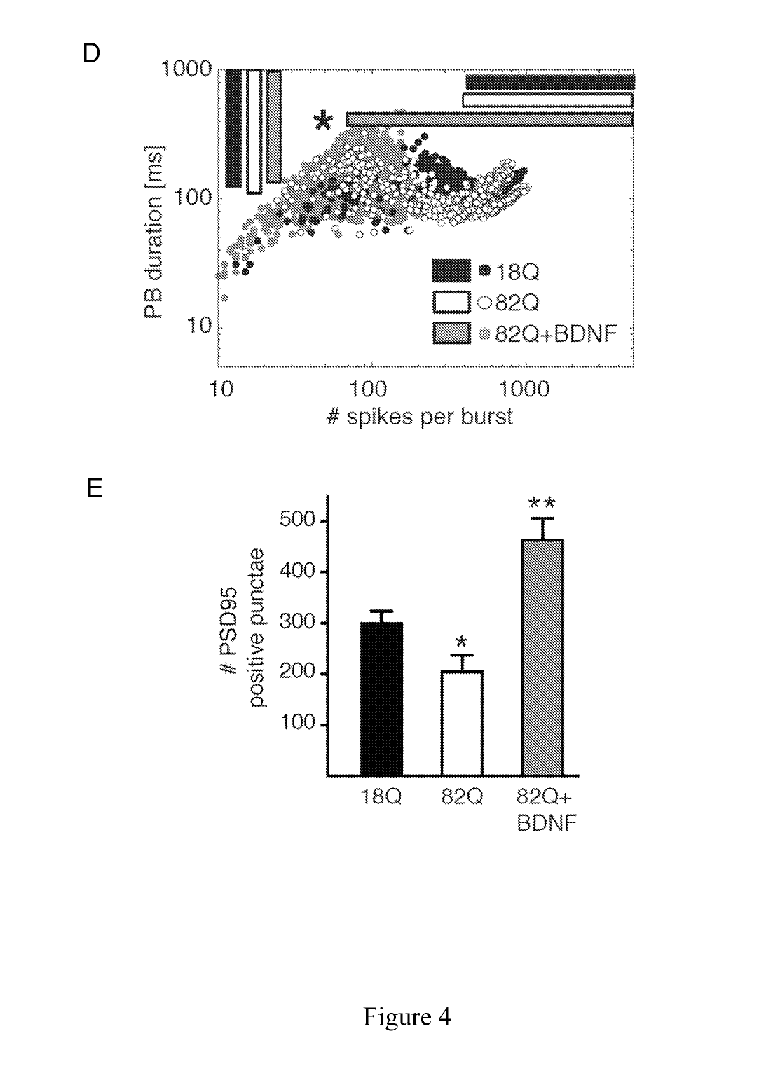

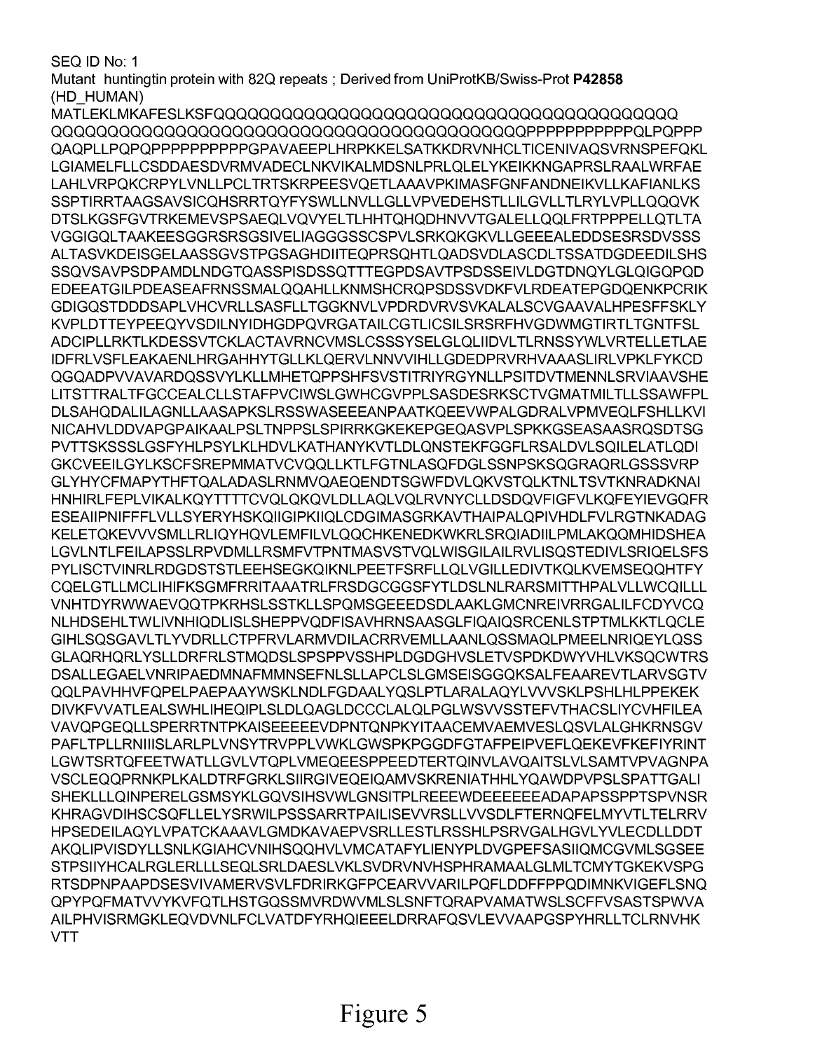

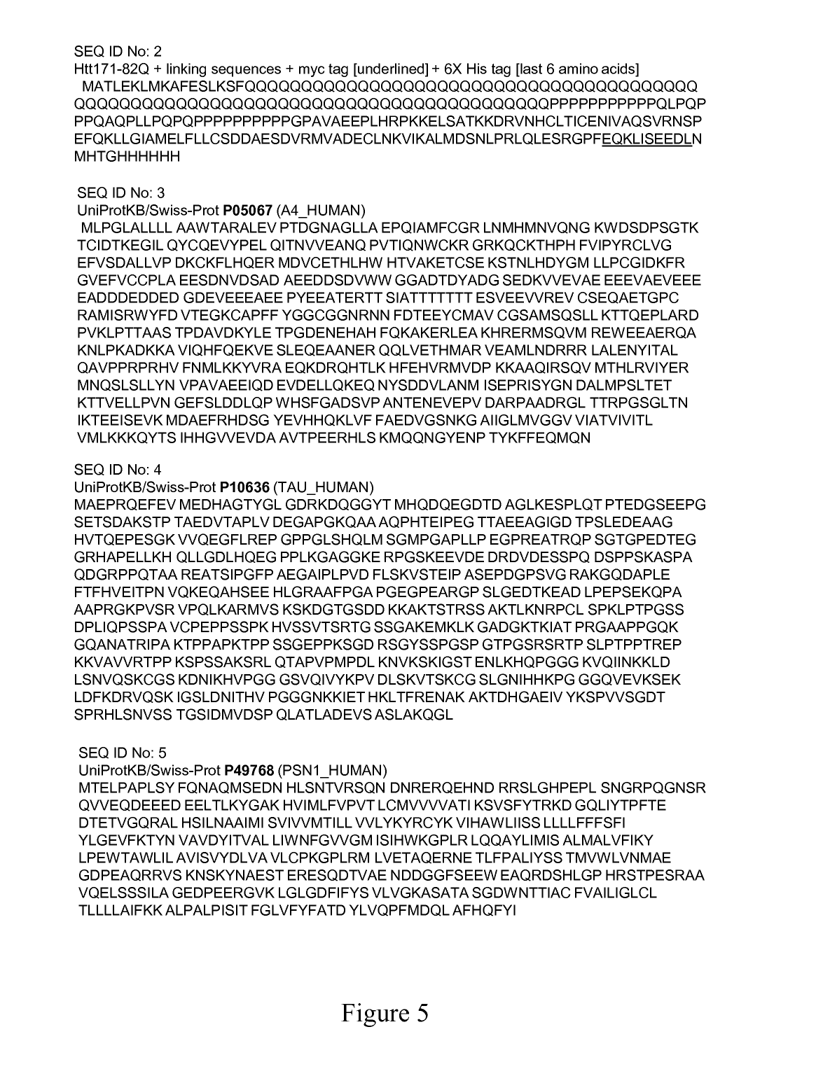

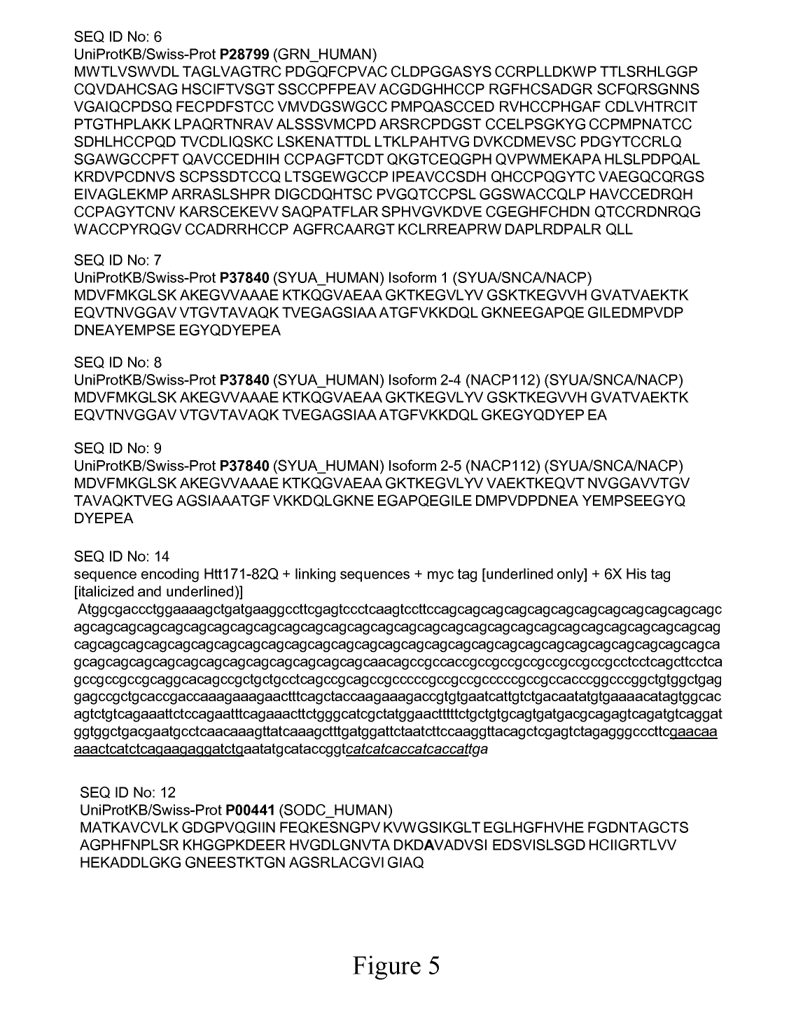

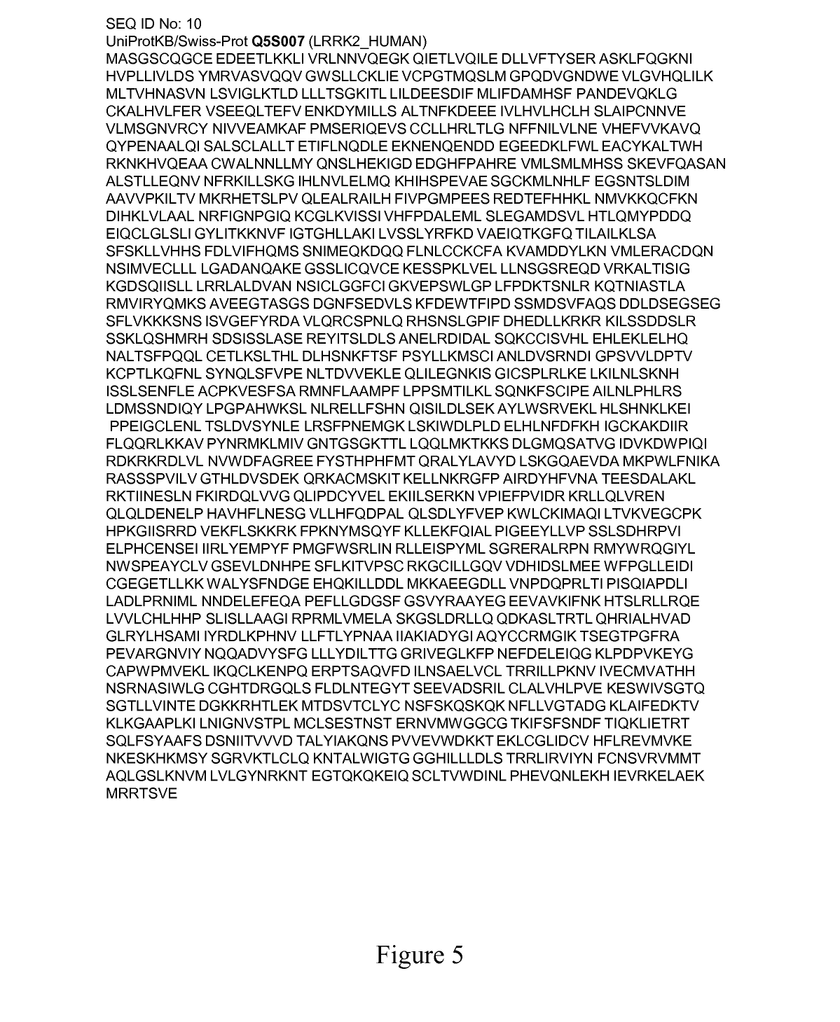

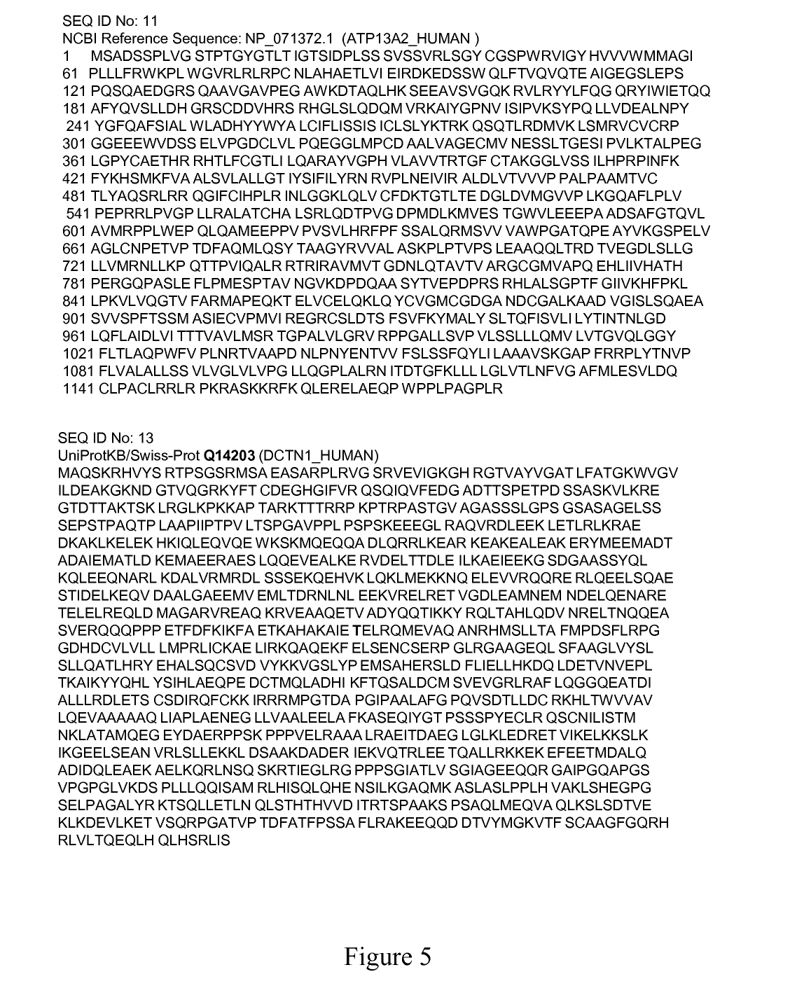

The earliest detected change was a deficit in activity-dependent induction of BDNF expression, which occurred at 10 days in vitro, i.e. prior to timepoints at which htt17′-82Q- and htt171-18Q-expressing cells could be differentiated on the basis of PB firing. These results suggested that a deficit in activity-dependent BDNF gene regulation might be responsible for diminished cortical microcircuit activity in HD cells. Further, exogenous addition BDNF to the culture medium was able to normalize the PB frequencies and IBI intervals of htt171-82Q-exposed cells, leading to significantly increased PB frequencies and decreased IBIs compared to non-BDNF-treated sister u) cultures (FIG. 4). Indeed, the restoring effect on PB timing by BDNF was readily apparent (FIG. 4A) and extended to both the IBI and duration distributions. Quantified in terms of numbers of individual action potentials, BDNF did not completely restore the size of individual PBs, however. Comparing samples of individual PBs recorded in BDNF treated and untreated htt171-82Q-exposed sister cells, their distribution is significantly shifted towards equally long PBs but slightly less populated by individual action potentials (FIG. 2E). Therefore, although BDNF normalized one major aspect of cortical microcircuit dysfunction (PB firing), it did not completely reverse polyQ htt effects. Specifically, the number of total spikes remained lower in htt171-82Q cells despite BDNF treatment. The lower spike number in htt17′-82Q cells also persisted despite a BDNF-mediated increase PSD95-positive boutons.

Taken together, these results support that decreased activity-dependent BDNF expression is a mediator of the cortical microcircuit hypoconnectivity in HD-affected cells.

The synaptic status was investigated in htt171-82Q- and htt171-18Q-expressing cells. Numbers of PSD95-positive structures was quantified as described above as an indicator of postsynaptic specializations, which comprise zones where neurotransmitter receptors, particularly glutamate receptors, are clustered. Htt171-82Q-expressing cells demonstrate a significant decrease in PSD95-positive bouton-like plasma membrane subdomains (punctae) which is reversed by BDNF treatment (FIG. 4E). These results suggest that the structural organization of synapses is one parameter of cortical microcircuit connectivity that may be affected by BDNF availability in HD cells.

The electrophysiological behaviours of htt17′-82Q-expressing neurons measured by the method according to the invention are in line with previous observations of HD brain: the observed reduced spike occurrence frequency may be attributed to abnormalities in voltage-gated sodium channels, intracellular calcium dynamics, potassium channels, and even toxic voltage-independent increased membrane permeability, all of which comprise previously identified HD-related phenomena. Further, the electrophysiological behaviours of htt17′-82Q-expressing neurons measured by the method according to the invention are consistent with observed impairments in cortical function in HD mice (Cepeda et al., 2007, J. Neurosci. Res., 78: 855-67) and significant reductions in the rate and synchrony of spontaneous burst firing observed in the cortices of HD mice compared to healthy mice by extracellular recordings in awake behaving animals (Walker et al., 2008, Neuroscience, 28: 8973-8982).

All together, these results support that a method according to the invention allows detection of electrophysiologic abnormalities linked to molecular alterations relevant for a disease-modifying effect and also allows the detection of the reversal of those abnormalities through exogeneous molecular supplementation to palliate those molecular alterations. Therefore, the implementation of engineered cortical cells for the present assay not only facilitates the study of patterned network activity (which arises spontaneously in excitatory neurons), but also provides a system complementary to striatal cells in which to examine and potentially remediate mutant htt's effects. Further, whereas the majority of currently used in vitro HD models are designed to examine cell survival or protein accumulation abnormalities, which may represent late stages of disease or be of uncertain relevance to addressing clinically meaningful endpoints, a method according to the invention provides as a simple, rapid and accurate way to probe neuronal network function upstream of neuronal cell death.

This further supports the use of a method according to the invention to screen substances on various excitable cellular models relevant for a wide range of neurological conditions, notably for substances capable to be active on early neuronal network-level dysfunction and/or capable of rescuing neuronal function.

### Sequence Listing of the Plasmid Sequences Encoding the Lentiviral Vectors

SEQ ID NO: 15:
SIN-TRE-Htt171-18Q-WPRE
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataag
gtagaagaggccaataaaggagagaacaccagcttgttacaccctgtgagcctgcatgggatggatgacccggagagagaa
gtgttagagtggaggtttgacagccgcctagcatttcatcacgtggcccgagagctgcatccggagtacttcaagaactgctga
tatcgagcttgctacaagggactttccgctggggactttccagggaggcgtggcctgggcgggactggggagtggcgagcc
ctcagatcctgcatataagcagctgctttttgcctgtactgggtctctctggttagaccagatctgagcctgggagctctctggcta
actagggaacccactgcttaagcctcaataaagcttgccttgagtgcttcaagtagtgtgtgcccgtctgttgtgtgactctggta
actagagatccctcagacccttttagtcagtgtggaaaatctctagcagtggcgcccgaacagggacctgaaagcgaaaggg
aaaccagagctctctcgacgcaggactcggcttgctgaagcgcgcgcacggcaagaggcgaggggcggcgactggtgag
tacgccaaaaattttgactagcggaggctagaaggagagagatgggtgcgagagcgtcagtattaagcgggggagaattag
atcgcgatgggaaaaaattcggttaaggccagggggaaagaaaaaatataaattaaaacatatagtatgggcaagcagggag
ctagaacgattcgcagttaatcctggcctgttagaaacatcagaaggctgtagacaaatactgggacagctacaaccatccctt
cagacaggatcagaagaacttagatcattatataatacagtagcaaccctctattgtgtgcatcaaaggatagagataaaagac
accaaggaagctttagacaagatagaggaagagcaaaacaaaagtaagaccaccgcacagcaagcggccgctgatcttca
gacctggaggaggagatatgagggacaattggagaagtgaattatataaatataaagtagtaaaaattgaaccattaggagta
gcacccaccaaggcaaagagaagagtggtgcagagagaaaaaagagcagtgggaataggagctttgttccttgggttcttg
ggagcagcaggaagcactatgggcgcagcgtcaatgacgctgacggtacaggccagacaattattgtctggtatagtgcagc
agcagaacaatttgctgagggctattgaggcgcaacagcatctgttgcaactcacagtctggggcatcaagcagctccaggc
aagaatcctggctgtggaaagatacctaaaggatcaacagctcctggggatttggggttgctctggaaaactcatttgcaccac
tgctgtgccttggaatgctagttggagtaataaatctctggaacagatttggaatcacacgacctggatggagtgggacagaga
aattaacaattacacaagcttaatacactccttaattgaagaatcgcaaaaccagcaagaaaagaatgaacaagaattattggaa
ttagataaatgggcaagtttgtggaattggtttaacataacaaattggctgtggtatataaaattattcataatgatagtaggaggct
tggtaggtttaagaatagtttttgctgtactttctatagtgaatagagttaggcagggatattcaccattatcgtttcagacccacctc
ccaaccccgaggggacccgacaggcccgaaggaatagaagaagaaggtggagagagagacagagacagatccattcga
ttagtgaacggatctcgacggtatcgatcacgagactagcctcgaccatcgatggtcgagtttaccactccctatcagtgataga
gaaaagtgaaagtcgagtttaccactccctatcagtgatagagaaaagtgaaagtcgagtttaccactccctatcagtgataga
gaaaagtgaaagtcgagtttaccactccctatcagtgatagagaaaagtgaaagtcgagtttaccactccctatcagtgataga
gaaaagtgaaagtcgagtttaccactccctatcagtgatagagaaaagtgaaagtcgagtttaccactccctatcagtgataga
gaaaagtgaaagtcgagctcggtacccgggtcgaggtaggcgtgtacggtgggaggcctatataagcagagctcgtttagtg
aaccgtcagatcgcctggagacgccatccacgctgttttgacctccatagaagacaccgggaccgatccagcctccgcggcc
ccgaattcgagctcggtacccggggatcaattctctagagatatcgtcgatggcgaccctggaaaagctgatgaaggccttcg
agtccctcaagtccttccagcagcagcagcagcagcagcagcagcagcagcagcagcagcagcagcaacagccgccacc
gccgccgccgccgccgccgccgccgccgcctcctcagcttcctcagccgccgccgcaggcacagccgctgctgcctcagc
cgcagccgcccccgccgccgcccccgccgccacccggcccggctgtggctgaggagccgctgcaccgaccaaagaaag
aactttcagctaccaagaaagaccgtgtgaatcattgtctgacaatatgtgaaaacatagtggcacagtctgtcagaaattctcca
gaatttcagaaacttctgggcatcgctatggaactttttctgctgtgcagtgatgacgcagagtcagatgtcaggatggtggctg
acgaatgcctcaacaaagttatcaaagctttgatggattctaatcttccaaggttacagctcgagtctagagggcccttcgaaca
aaaactcatctcagaagaggatctgaatatgcataccggtcatcatcaccatcaccattgagttttcgagtgagagaagattttca
gcctgatacagattaaaatcgtcgagggaattgatcctctagagtcgacctgcaggcatgcaagctaattccgataatcaacctc
tggattacaaaatttgtgaaagattgactggtattcttaactatgttgctccttttacgctatgtggatacgctgctttaatgcctttgta
tcatgctattgcttcccgtatggctttcattttctcctccttgtataaatcctggttgctgtctctttatgaggagttgtggcccgttgtca
ggcaacgtggcgtggtgtgcactgtgtttgctgacgcaacccccactggttggggcattgccaccacctgtcagctcctttccg
ggactttcgctttccccctccctattgccacggcggaactcatcgccgcctgccttgcccgctgctggacaggggctcggctgt
tgggcactgacaattccgtggtgttgtcggggaagctgacgtcctttccatggctgctcgcctgtgttgccacctggattctgcg
cgggacgtccttctgctacgtcccttcggccctcaatccagcggaccttccttcccgcggcctgctgccggctctgcggcctctt
ccgcgtcttcgccttcgccctcagacgagtcggatctccctttgggccgcctccccgcatcgggaattagcttgttaacatcgat
ggaattcgagctcggtacctttaaaaccaatgacttacaaggcagctgtaaatcttagccactttttaaaagaaaaggggggact
ggaagggctaattcactcccaacgaaaacaaaatctgctttttgcttgtactgggtctctctggttagaccaaatctgagcctggg
agctctctggctaactagggaacccactgcttaagcctcaataaagcttgccttgagtgcttcaagtagtgtgtgcccgtctgttg
tgtgactctggtaactagagatccctcaaacccttttagtcagtgtggaaaatctctagcagcatctagaattaattccgtgtattct
atagtgtcacctaaatcgtatgtgtatgatacataaggttatgtattaattgtagccgcgttctaacgacaatatgtacaagcctaatt
gtgtagcatctggcttactgaagcagaccctatcatctctctcgtaaactgccgtcagagtcggtttggttggacgaaccttctga
gtttctggtaacgccgtcccgcacccggaaatggtcagcgaaccaatcagcagggtcatcgctagccagatcctctacgccg
gacgcatcgtggccggcatcaccggcgccacaggtgcggttgctggcgcctatatcgccgacatcaccgatggggaagatc
gggctcgccacttcgggctcatgagcgcttgtttcggcgtgggtatggtggcaggccccgtggccgggggactgttgggcgc
catctccttgcatgcaccattccttgcggcggcggtgctcaacggcctcaacctactactgggctgcttcctaatgcaggagtcg
cataagggagagcgtcgatatggtgcactctcagtacaatctgctctgatgccgcatagttaagccagccccgacacccgcca
acacccgctgacgcgccctgacgggcttgtctgctcccggcatccgcttacagacaagctgtgaccgtctccgggagctgca
tgtgtcagaggttttcaccgtcatcaccgaaacgcgcgagacgaaagggcctcgtgatacgcctatttttataggttaatgtcatg
ataataatggtttcttagacgtcaggtggcacttttcggggaaatgtgcgcggaacccctatttgtttatttttctaaatacattcaaa
tatgtatccgctcatgagacaataaccctgataaatgcttcaataatattgaaaaaggaagagtatgagtattcaacatttccgtgt
cgcccttattcccttttttgcggcattttgccttcctgtttttgctcacccagaaacgctggtgaaagtaaaagatgctgaagatcag
ttgggtgcacgagtgggttacatcgaactggatctcaacagcggtaagatccttgagagttttcgccccgaagaacgttttccaa
tgatgagcacttttaaagttctgctatgtggcgcggtattatcccgtattgacgccgggcaagagcaactcggtcgccgcataca
ctattctcagaatgacttggttgagtactcaccagtcacagaaaagcatcttacggatggcatgacagtaagagaattatgcagt
gctgccataaccatgagtgataacactgcggccaacttacttctgacaacgatcggaggaccgaaggagctaaccgcttttttg
cacaacatgggggatcatgtaactcgccttgatcgttgggaaccggagctgaatgaagccataccaaacgacgagcgtgaca
ccacgatgcctgtagcaatggcaacaacgttgcgcaaactattaactggcgaactacttactctagcttcccggcaacaattaat
agactggatggaggcggataaagttgcaggaccacttctgcgctcggcccttccggctggctggtttattgctgataaatctgg
agccggtgagcgtgggtctcgcggtatcattgcagcactggggccagatggtaagccctcccgtatcgtagttatctacacga
cggggagtcaggcaactatggatgaacgaaatagacagatcgctgagataggtgcctcactgattaagcattggtaactgtca
gaccaagtttactcatatatactttagattgatttaaaacttcatttttaatttaaaaggatctaggtgaagatcctttttgataatctcat
gaccaaaatcccttaacgtgagttttcgttccactgagcgtcagaccccgtagaaaagatcaaaggatcttcttgagatcctttttt
tctgcgcgtaatctgctgcttgcaaacaaaaaaaccaccgctaccagcggtggtttgtttgccggatcaagagctaccaactctt
tttccgaaggtaactggcttcagcagagcgcagataccaaatactgtccttctagtgtagccgtagttaggccaccacttcaaga
actctgtagcaccgcctacatacctcgctctgctaatcctgttaccagtggctgctgccagtggcgataagtcgtgtcttaccgg
gttggactcaagacgatagttaccggataaggcgcagcggtcgggctgaacggggggttcgtgcacacagcccagcttgga
gcgaacgacctacaccgaactgagatacctacagcgtgagctatgagaaagcgccacgcttcccgaagggagaaaggcgg
acaggtatccggtaagcggcagggtcggaacaggagagcgcacgagggagcttccagggggaaacgcctggtatctttat
agtcctgtcgggtttcgccacctctgacttgagcgtcgatttttgtgatgctcgtcaggggggcggagcctatggaaaaacgcc
agcaacgcggcctttttacggttcctggccttttgctggccttttgctcacatgttctttcctgcgttatcccctgattctgtggataac
cgtattaccgcctttgagtgagctgataccgctcgccgcagccgaacgaccgagcgcagcgagtcagtgagcgaggaagc
ggaagagcgcccaatacgcaaaccgcctctccccgcgcgttggccgattcattaatgcagctgtggaatgtgtgtcagttagg
gtgtggaaagtccccaggctccccagcaggcagaagtatgcaaagcatgcatctcaattagtcagcaaccaggtgtggaaag
tccccaggctccccagcaggcagaagtatgcaaagcatgcatctcaattagtcagcaaccatagtcccgcccctaactccgcc
catcccgcccctaactccgcccagttccgcccattctccgccccatggctgactaattttttttatttatgcagaggccgaggccg
cctcggcctctgagctattccagaagtagtgaggaggcttttttggaggcctaggcttttgcaaaaagcttggacacaagacag
gcttgcgagatatgtttgagaataccactttatcccgcgtcagggagaggcagtgcgtaaaaagacgcggactcatgtgaaat
actggtttttagtgcgccagatctctataatctcgcgcaacctattttcccctcgaacactttttaagccgtagataaacaggctgg
gacacttcacatgagcgaaaaatacatcgtcacctgggacatgttgcagatccatgcacgtaaactcgcaagccgactgatgc
cttctgaacaatggaaaggcattattgccgtaagccgtggcggtctgtaccgggtgcgttactggcgcgtgaactgggtattcg
tcatgtcgataccgtttgtatttccagctacgatcacgacaaccagcgcgagcttaaagtgctgaaacgcgcagaaggcgatg
gcgaaggcttcatcgttattgatgacctggtggataccggtggtactgcggttgcgattcgtgaaatgtatccaaaagcgcacttt
gtcaccatcttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgttgttcttttta
acttcaggcgggttacaatagtttccagtaagtattctggaggctgcatccatgacacaggcaaacctgagcgaaaccctgttc
aaaccccgctttaaacatcctgaaacctcgacgctagtccgccgctttaatcacggcgcacaaccgcctgtgcagtcggccctt
gatggtaaaaccatccctcactggtatcgcatgattaaccgtctgatgtggatctggcgcggcattgacccacgcgaaatcctc
gacgtccaggcacgtattgtgatgagcgatgccgaacgtaccgacgatgatttatacgatacggtgattggctaccgtggcgg
caactggatttatgagtgggccccggatctttgtgaaggaaccttacttctgtggtgtgacataattggacaaactacctacagag
atttaaagctctaaggtaaatataaaatttttaagtgtataatgtgttaaactactgattctaattgtttgtgtattttagattccaacctat
ggaactgatgaatgggagcagtggtggaatgcctttaatgaggaaaacctgttttgctcagaagaaatgccatctagtgatgat
gaggctactgctgactctcaacattctactcctccaaaaaagaagagaaaggtagaagaccccaaggactttccttcagaattg
ctaagttttttgagtcatgctgtgtttagtaatagaactcttgcttgctttgctatttacaccacaaaggaaaaagctgcactgctata
caagaaaattatggaaaaatattctgtaacctttataagtaggcataacagttataatcataacatactgttttttcttactccacaca
ggcatagagtgtctgctattaataactatgctcaaaaattgtgtacctttagctttttaatttgtaaaggggttaataaggaatatttga
tgtatagtgccttgactagagatcataatcagccataccacatttgtagagcttttacttgctttaaaaaacctcccacacctccccc
tgaacctgaaacataaaatgaatgcaattgttgttgttaacttgtttattgcagcttataatggttacaaataaagcaatagcatcac
aaatttcacaaataaagcatttttttcactgcattctagttgtggtttgtccaaactcatcaatgtatcttatcatgtgtggatcaactgg
ataactcaagctaaccaaaatcatcccaaacttcccaccccataccctattaccactgccaaattacctgtggtttcatttactctaa
acctgtgattcctctgaattattttcattttaaagaaattgtatttgttaaatatgtactacaaacttagtagt

Feature map:

Htt171-18Q: 2545-3051; 5′LTR: 1-634; TRE: 2055-2366; Myc-tag: 3067-3096; HIS-tag: 3112-3129; WPRE: 3224-3831; 3′LTR: 3933-4166; SV40 polyA: 8356-9205; P min CMV: 2368-2487; TATA: 2390-2397; cPPT: 3916-3930.

SEQ ID NO: 16:
SIN-TRE-Htt171-82Q-WPRE
tggaagggctaattcactcccaaagaagacaagatatccttgatctgtggatctaccacacacaaggctacttccctgattagca
gaactacacaccagggccaggggtcagatatccactgacctttggatggtgctacaagctagtaccagttgagccagataag
gtagaagaggccaataaaggagagaacaccagcttgttacaccctgtgagcctgcatgggatggatgacccggagagagaa
gtgttagagtggaggtttgacagccgcctagcatttcatcacgtggcccgagagctgcatccggagtacttcaagaactgctga
tatcgagcttgctacaagggactttccgctggggactttccagggaggcgtggcctgggcgggactggggagtggcgagcc
ctcagatcctgcatataagcagctgctttttgcctgtactgggtctctctggttagaccagatctgagcctgggagctctctggcta
actagggaacccactgcttaagcctcaataaagcttgccttgagtgcttcaagtagtgtgtgcccgtctgttgtgtgactctggta
actagagatccctcagacccttttagtcagtgtggaaaatctctagcagtggcgcccgaacagggacctgaaagcgaaaggg
aaaccagagctctctcgacgcaggactcggcttgctgaagcgcgcgcacggcaagaggcgaggggcggcgactggtgag
tacgccaaaaattttgactagcggaggctagaaggagagagatgggtgcgagagcgtcagtattaagcgggggagaattag
atcgcgatgggaaaaaattcggttaaggccagggggaaagaaaaaatataaattaaaacatatagtatgggcaagcagggag
ctagaacgattcgcagttaatcctggcctgttagaaacatcagaaggctgtagacaaatactgggacagctacaaccatccctt
cagacaggatcagaagaacttagatcattatataatacagtagcaaccctctattgtgtgcatcaaaggatagagataaaagac
accaaggaagctttagacaagatagaggaagagcaaaacaaaagtaagaccaccgcacagcaagcggccgctgatcttca
gacctggaggaggagatatgagggacaattggagaagtgaattatataaatataaagtagtaaaaattgaaccattaggagta
gcacccaccaaggcaaagagaagagtggtgcagagagaaaaaagagcagtgggaataggagctttgttccttgggttcttg
ggagcagcaggaagcactatgggcgcagcgtcaatgacgctgacggtacaggccagacaattattgtctggtatagtgcagc
agcagaacaatttgctgagggctattgaggcgcaacagcatctgttgcaactcacagtctggggcatcaagcagctccaggc
aagaatcctggctgtggaaagatacctaaaggatcaacagctcctggggatttggggttgctctggaaaactcatttgcaccac
tgctgtgccttggaatgctagttggagtaataaatctctggaacagatttggaatcacacgacctggatggagtgggacagaga
aattaacaattacacaagcttaatacactccttaattgaagaatcgcaaaaccagcaagaaaagaatgaacaagaattattggaa
ttagataaatgggcaagtttgtggaattggtttaacataacaaattggctgtggtatataaaattattcataatgatagtaggaggct
tggtaggtttaagaatagtttttgctgtactttctatagtgaatagagttaggcagggatattcaccattatcgtttcagacccacctc
ccaaccccgaggggacccgacaggcccgaaggaatagaagaagaaggtggagagagagacagagacagatccattcga
ttagtgaacggatctcgacggtatcgatcacgagactagcctcgaccatcgatggtcgagtttaccactccctatcagtgataga
gaaaagtgaaagtcgagtttaccactccctatcagtgatagagaaaagtgaaagtcgagtttaccactccctatcagtgataga
gaaaagtgaaagtcgagtttaccactccctatcagtgatagagaaaagtgaaagtcgagtttaccactccctatcagtgataga
gaaaagtgaaagtcgagtttaccactccctatcagtgatagagaaaagtgaaagtcgagtttaccactccctatcagtgataga
gaaaagtgaaagtcgagctcggtacccgggtcgaggtaggcgtgtacggtgggaggcctatataagcagagctcgtttagtg
aaccgtcagatcgcctggagacgccatccacgctgttttgacctccatagaagacaccgggaccgatccagcctccgcggcc
ccgaattcgagctcggtacccggggatcaattctctagagatatcgtcgacatggcgaccctggaaaagctgatgaaggcctt
cgagtccctcaagtccttccagcagcagcagcagcagcagcagcagcagcagcagcagcagcagcagcagcagcagca
gcagcagcagcagcagcagcagcagcagcagcagcagcagcagcagcagcagcagcagcagcagcagcagcagcag
cagcagcagcagcagcagcagcagcagcagcagcagcagcagcagcagcagcagcagcagcagcagcagcagcagc
agcagcagcagcagcagcagcagcagcagcaacagccgccaccgccgccgccgccgccgccgcctcctcagcttcctca
gccgccgccgcaggcacagccgctgctgcctcagccgcagccgcccccgccgccgcccccgccgccacccggcccggc
tgtggctgaggagccgctgcaccgaccaaagaaagaactttcagctaccaagaaagaccgtgtgaatcattgtctgacaatat
gtgaaaacatagtggcacagtctgtcagaaattctccagaatttcagaaacttctgggcatcgctatggaactttttctgctgtgca
gtgatgacgcagagtcagatgtcaggatggtggctgacgaatgcctcaacaaagttatcaaagctttgatggattctaatcttcc
aaggttacagctcgagtctagagggcccttcgaacaaaaactcatctcagaagaggatctgaatatgcataccggtcatcatca
ccatcaccattgagttttcgagtgagagaagattttcagcctgatacagattaaaatcgtcgagggaattgatcctctagagtcga
cctgcaggcatgcaagctaattccgataatcaacctctggattacaaaatttgtgaaagattgactggtattcttaactatgttgctc
cttttacgctatgtggatacgctgctttaatgcctttgtatcatgctattgcttcccgtatggctttcattttctcctccttgtataaatcct
ggttgctgtctctttatgaggagttgtggcccgttgtcaggcaacgtggcgtggtgtgcactgtgtttgctgacgcaacccccact
ggttggggcattgccaccacctgtcagctcctttccgggactttcgctttccccctccctattgccacggcggaactcatcgccg
cctgccttgcccgctgctggacaggggctcggctgttgggcactgacaattccgtggtgttgtcggggaagctgacgtcctttc
catggctgctcgcctgtgttgccacctggattctgcgcgggacgtccttctgctacgtcccttcggccctcaatccagcggacct
tccttcccgcggcctgctgccggctctgcggcctcttccgcgtcttcgccttcgccctcagacgagtcggatctccctttgggcc
gcctccccgcatcgggaattagcttgttaacatcgatggaattcgagctcggtacctttaaaaccaatgacttacaaggcagctg
taaatcttagccactttttaaaagaaaaggggggactggaagggctaattcactcccaacgaaaacaaaatctgctttttgcttgt
actgggtctctctggttagaccaaatctgagcctgggagctctctggctaactagggaacccactgcttaagcctcaataaagct
tgccttgagtgcttcaagtagtgtgtgcccgtctgttgtgtgactctggtaactagagatccctcaaacccttttagtcagtgtgga
aaatctctagcagcatctagaattaattccgtgtattctatagtgtcacctaaatcgtatgtgtatgatacataaggttatgtattaatt
gtagccgcgttctaacgacaatatgtacaagcctaattgtgtagcatctggcttactgaagcagaccctatcatctctctcgtaaa
ctgccgtcagagtcggtttggttggacgaaccttctgagtttctggtaacgccgtcccgcacccggaaatggtcagcgaacca
atcagcagggtcatcgctagccagatcctctacgccggacgcatcgtggccggcatcaccggcgccacaggtgcggttgct
ggcgcctatatcgccgacatcaccgatggggaagatcgggctcgccacttcgggctcatgagcgcttgtttcggcgtgggtat
ggtggcaggccccgtggccgggggactgttgggcgccatctccttgcatgcaccattccttgcggcggcggtgctcaacgg
cctcaacctactactgggctgcttcctaatgcaggagtcgcataagggagagcgtcgatatggtgcactctcagtacaatctgct
ctgatgccgcatagttaagccagccccgacacccgccaacacccgctgacgcgccctgacgggcttgtctgctcccggcatc
cgcttacagacaagctgtgaccgtctccgggagctgcatgtgtcagaggttttcaccgtcatcaccgaaacgcgcgagacga
aagggcctcgtgatacgcctatttttataggttaatgtcatgataataatggtttcttagacgtcaggtggcacttttcggggaaatg
tgcgcggaacccctatttgtttatttttctaaatacattcaaatatgtatccgctcatgagacaataaccctgataaatgcttcaataat
attgaaaaaggaagagtatgagtattcaacatttccgtgtcgcccttattcccttttttgcggcattttgccttcctgtttttgctcacc
cagaaacgctggtgaaagtaaaagatgctgaagatcagttgggtgcacgagtgggttacatcgaactggatctcaacagcgg
taagatccttgagagttttcgccccgaagaacgttttccaatgatgagcacttttaaagttctgctatgtggcgcggtattatcccgt
attgacgccgggcaagagcaactcggtcgccgcatacactattctcagaatgacttggttgagtactcaccagtcacagaaaa
gcatcttacggatggcatgacagtaagagaattatgcagtgctgccataaccatgagtgataacactgcggccaacttacttctg
acaacgatcggaggaccgaaggagctaaccgcttttttgcacaacatgggggatcatgtaactcgccttgatcgttgggaacc
ggagctgaatgaagccataccaaacgacgagcgtgacaccacgatgcctgtagcaatggcaacaacgttgcgcaaactatta
actggcgaactacttactctagettcccggcaacaattaatagactggatggaggcggataaagttgcaggaccacttctgcgc
tcggcccttccggctggctggtttattgctgataaatctggagccggtgagcgtgggtctcgcggtatcattgcagcactgggg
ccagatggtaagccctcccgtatcgtagttatctacacgacggggagtcaggcaactatggatgaacgaaatagacagatcgc
tgagataggtgcctcactgattaagcattggtaactgtcagaccaagtttactcatatatactttagattgatttaaaacttcattttta
atttaaaaggatctaggtgaagatcctttttgataatctcatgaccaaaatcccttaacgtgagttttcgttccactgagcgtcagac
cccgtagaaaagatcaaaggatcttcttgagatcctttttttctgcgcgtaatctgctgcttgcaaacaaaaaaaccaccgctacc
agcggtggtttgtttgccggatcaagagctaccaactctttttccgaaggtaactggcttcagcagagcgcagataccaaatact
gtccttctagtgtagccgtagttaggccaccacttcaagaactctgtagcaccgcctacatacctcgctctgctaatcctgttacc
agtggctgctgccagtggcgataagtcgtgtcttaccgggttggactcaagacgatagttaccggataaggcgcagcggtcg
ggctgaacggggggttcgtgcacacagcccagcttggagcgaacgacctacaccgaactgagatacctacagcgtgagcta
tgagaaagcgccacgcttcccgaagggagaaaggcggacaggtatccggtaagcggcagggtcggaacaggagagcgc
acgagggagettccagggggaaacgcctggtatctttatagtcctgtcgggtttcgccacctctgacttgagcgtcgatttttgtg
atgctcgtcaggggggcggagcctatggaaaaacgccagcaacgcggcctttttacggttcctggccttttgctggccttttgct
cacatgttctttcctgcgttatcccctgattctgtggataaccgtattaccgcctttgagtgagctgataccgctcgccgcagccga
acgaccgagcgcagcgagtcagtgagcgaggaagcggaagagcgcccaatacgcaaaccgcctctccccgcgcgttggc
cgattcattaatgcagctgtggaatgtgtgtcagttagggtgtggaaagtccccaggctccccagcaggcagaagtatgcaaa
gcatgcatctcaattagtcagcaaccaggtgtggaaagtccccaggctccccagcaggcagaagtatgcaaagcatgcatct
caattagtcagcaaccatagtcccgcccctaactccgcccatcccgcccctaactccgcccagttccgcccattctccgcccca
tggctgactaattttttttatttatgcagaggccgaggccgcctcggcctctgagctattccagaagtagtgaggaggcttttttgg
aggcctaggcttttgcaaaaagcttggacacaagacaggcttgcgagatatgtttgagaataccactttatcccgcgtcaggga
gaggcagtgcgtaaaaagacgcggactcatgtgaaatactggtttttagtgcgccagatctctataatctcgcgcaacctattttc
ccctcgaacactttttaagccgtagataaacaggctgggacacttcacatgagcgaaaaatacatcgtcacctgggacatgttg
cagatccatgcacgtaaactcgcaagccgactgatgccttctgaacaatggaaaggcattattgccgtaagccgtggcggtct
gtaccgggtgcgttactggcgcgtgaactgggtattcgtcatgtcgataccgtttgtatttccagctacgatcacgacaaccagc
gcgagcttaaagtgctgaaacgcgcagaaggcgatggcgaaggcttcatcgttattgatgacctggtggataccggtggtact
gcggttgcgattcgtgaaatgtatccaaaagcgcactttgtcaccatcttcgcaaaaccggctggtcgtccgctggttgatgact
atgttgttgatatcccgcaagatacctggattgaacagccgtgggatatgggcgtcgtattcgtcccgccaatctccggtcgcta
atcttttcaacgcctggcactgccgggcgttgttctttttaacttcaggcgggttacaatagtttccagtaagtattctggaggctgc
atccatgacacaggcaaacctgagcgaaaccctgttcaaaccccgctttaaacatcctgaaacctcgacgctagtccgccgctt
taatcacggcgcacaaccgcctgtgcagtcggcccttgatggtaaaaccatccctcactggtatcgcatgattaaccgtctgat
gtggatctggcgcggcattgacccacgcgaaatcctcgacgtccaggcacgtattgtgatgagcgatgccgaacgtaccgac
gatgatttatacgatacggtgattggctaccgtggeggcaactggatttatgagtgggccccggatctttgtgaaggaaccttac
ttctgtggtgtgacataattggacaaactacctacagagatttaaagctctaaggtaaatataaaatttttaagtgtataatgtgttaa
actactgattctaattgtttgtgtattttagattccaacctatggaactgatgaatgggagcagtggtggaatgcctttaatgaggaa
aacctgttttgctcagaagaaatgccatctagtgatgatgaggctactgctgactctcaacattctactcctccaaaaaagaagag
aaaggtagaagaccccaaggactttccttcagaattgctaagttttttgagtcatgctgtgtttagtaatagaactcttgcttgctttg
ctatttacaccacaaaggaaaaagctgcactgctatacaagaaaattatggaaaaatattctgtaacctttataagtaggcataac
agttataatcataacatactgttttttcttactccacacaggcatagagtgtctgctattaataactatgctcaaaaattgtgtaccttta
gctttttaatttgtaaaggggttaataaggaatatttgatgtatagtgccttgactagagatcataatcagccataccacatttgtaga
gcttttacttgctttaaaaaacctcccacacctccccctgaacctgaaacataaaatgaatgcaattgttgttgttaacttgtttattgc
agcttataatggttacaaataaagcaatagcatcacaaatttcacaaataaagcatttttttcactgcattctagttgtggtttgtcca
aactcatcaatgtatcttatcatgtgtggatcaactggataactcaagctaaccaaaatcatcccaaacttcccaccccatacccta
ttaccactgccaaattacctgtggtttcatttactctaaacctgtgattcctctgaattattttcattttaaagaaattgtatttgttaaata
tgtactacaaacttagtagt

Feature map:

Htt171-82Q: 2547-3236; 5′LTR: 1-634; TRE: 2055-2366; Myc-tag: 3252-3281; HIS-tag: 3297-3314; WPRE: 3409-4016; 3′LTR: 4118-4351; P min CMV: 2368-2487; TATA: 2390-2397; cPPT: 4101-4115.

1-13. (canceled)

14. An ex-vivo method for assaying a neuroactive substance comprising:
(i) providing a neuronal cell culture sample comprising genetically engineered neuronal cells wherein neuronal cells are genetically engineered to express at least one neurodegeneratively active protein;
(ii) comparing at least one electrophysiological response parameter measured simultaneously at a plurality of regions in said neuronal cell culture sample when contacted with a candidate substance or a candidate substance composition with a baseline electrophysiological response parameter of said regions;
(iii) determining the difference between said electrophysiological response parameter and said baseline electrophysiological response parameter; and
(iv) detecting the presence or absence of a neuroactive substance in said candidate substance or a candidate substance composition based upon the difference determined under step (iii) and/or detecting a neuronal adverse effect of said candidate substance or candidate substance composition based upon the difference determined under step (iii).

15. The method according to claim 14, wherein the neurodegeneratively active protein is a mutant of huntingtin protein (SEQ ID NO: 1) or a variant or a fragment thereof.

16. The method according to claim 14, wherein the neurodegeneratively active protein is selected from the group consisting of: the full-length amyloid precursor protein (APP) (SEQ ID NO: 3) or a variant thereof or a fragment thereof, microtubule-associated protein tau (MAPT) (SEQ ID NO: 4) or a variant or a fragment thereof, full-length presenilin 1 (PSEN1) (SEQ ID NO: 5), or a variant thereof or a fragment thereof, granulin (GRN) (SEQ ID NO: 6) or a variant thereof or a fragment thereof, alpha-synuclein (SNCA) isoform 1 (SEQ ID NO: 7), isoform 2-4 (SEQ ID NO: 8), isoform 2-5 (SEQ ID NO: 9) or a variant thereof or a fragment thereof, leucine-rich repeat kinase 2 (LRRK2) (SEQ ID NO: 10) or a variant thereof or a fragment thereof, ATPase type 13A2 (ATP 13A2) (SEQ ID NO: 11) or a variant thereof or a fragment thereof, superoxide dismutase 1 (SOD1) (SEQ ID NO: 12) or a variant thereof or a fragment thereof, and dynactin 1 (DCTN1) (SEQ ID NO: 13) or a variant thereof or a fragment thereof.

17. The method according to claim 14, wherein the genetically engineered neuronal cells are produced by a method comprising a step of contacting a neuronal cell with a sell inactivating HIV1-derived lentiviral vector comprising a nucleic acid sequence encoding said neurodegeneratively active protein operably linked to at least one sequence which controls expression of the corresponding protein.

18. The method according to claim 14, wherein the neurodegeneratively active protein is a mutant huntingtin fragment of SEQ ID NO: 2.

19. The method according to claim 14, wherein said neurodegeneratively active protein is selected from the group consisting of: a variant of the full-length amyloid precursor protein (APP) (SEQ ID NO: 3) consisting of APP with missense mutation KM/670/671/NL or missense mutation E693G or missense mutation V717F or a fragment thereof, a variant of a microtubule-associated protein tau (MAPT) (SEQ ID NO: 4) consisting of MAPT with missense mutation L618P or a fragment thereof, a variant of full-length presenilin 1 (PSEN1) (SEQ ID NO: 5) consisting of PSEN1 with missense mutation V82L or missense mutation V96F or deletion IM 83/84 or a fragment thereof, a variant of granulin (GRN) (SEQ ID NO: 6) or a fragment thereof, a variant of alpha-synuclein (SNCA) isoform 1 (SEQ ID NO: 7), isoform 2-4 (SEQ ID NO: 8), isoform 2-5 (SEQ ID NO: 9) or a fragment thereof, a variant of leucine-rich repeat kinase 2 (LRRK2) (SEQ ID NO: 10) consisting of LRRK2 with missense mutation G2019S or a fragment thereof, a variant of ATPase type 13A2 (ATP 13A2) (SEQ ID NO: 11) consisting of ATP 13A2 with missense mutation G504R or a fragment thereof, a variant of superoxide dismutase 1 (SOD1) (SEQ ID NO: 12) consisting of SOD1 with missense mutation G93A or a fragment thereof, and a variant of dynactin 1 (DCTN1) (SEQ ID NO: 13) consisting of DCTN1 with missense mutation M571T or a fragment thereof.

20. The method according to claim 14, wherein the genetically engineered neuronal cells are produced by a method comprising a step of contacting a neuronal cell with a self-inactivating HIV1-derived lentiviral vector comprising SEQ ID NO: 16.

21. The method according to claim 14, wherein step (iii) comprises a data processing step that can be implemented by a computer to analyze changes in at least one action potential characteristics of the cells upon exposure to the candidate substance or candidate substance composition.

22. The method according to claim 14, wherein the measurement of the said at least one electrophysiological response parameter is coupled with the recording of at least one morphological and/or structural parameter of the neuronal cells.

23. The method according to claim 14, wherein said at least one electrophysiological response parameter is measured by extracellular multielectrode array.

24. An isolated genetically engineered neuronal cell for the electrophysiological detection of a neuroactive substance wherein the neuronal cells are genetically engineered to express a neurodegeneratively active protein selected from the group consisting of: a variant of the full-length amyloid precursor protein (APP) (SEQ ID NO: 3) consisting of APP with missense mutation KM/670/671/NL or missense mutation E693G or missense mutation V717F or a fragment thereof, a variant of a microtubule-associated protein tau (MAPT) (SEQ ID NO: 4) consisting of MAPT with missense mutation L618P or a fragment thereof, a variant of full-length presenilin 1 (PSEN1) (SEQ ID NO: 5) consisting of PSEN1 with missense mutation V82L or missense mutation V96F or deletion IM 83/84 or a fragment thereof, granulin (GRN) (SEQ ID NO: 6) or a variant thereof or a fragment thereof, alpha-synuclein (SNCA) isoform 1 (SEQ ID NO: 7), isoform 2-4 (SEQ ID NO: 8), isoform 2-5 (SEQ ID NO: 9) or a variant thereof or a fragment thereof, ATPase type 13A2 (ATP13A2) (SEQ ID NO: 11) or a variant thereof or a fragment thereof, and dynactin 1 (DCTN1) (SEQ ID NO: 13) or a variant thereof or a fragment thereof.

25. The isolated genetically engineered neuronal cell according to claim 24, wherein said neurodegeneratively active protein is selected from the group consisting of: a variant of ATPase type 13A2 (ATP13A2) (SEQ ID NO: 11) consisting of ATP13A2 with missense mutation G504R or a fragment thereof, and a variant of dynactin 1 (DCTN1) (SEQ ID NO: 13) consisting of DCTN1 with missense mutation M571T or a fragment thereof.

26. An isolated neuronal cell composition comprising at least one cell isolated according to claim 24.

27. A kit for electrophysiological detection of a neuroactive substance, the kit comprising genetically engineered neuronal cells or a composition thereof or neuronal cells together with vectors to genetically engineer neuronal cells, wherein the genetically engineered neuronal cells or the neuronal cells once transduced with the said vectors express a neurodegeneratively active protein.

28. The kit according to claim 27, wherein the neurodegeneratively active protein is a mutant of huntingtin protein (SEQ ID NO: 1) or a variant or a fragment thereof.

29. The kit according to claim 27, wherein the neurodegeneratively active protein is a mutant huntingtin fragment of SEQ ID NO: 2.

30. The kit according to claim 27, wherein the neurodegeneratively active protein is selected from the group consisting of: the full-length amyloid precursor protein (APP) (SEQ ID NO: 3) or a variant thereof or a fragment thereof, microtubule-associated protein tau (MAPT) (SEQ ID NO: 4) or a variant or a fragment thereof, full-length presenilin 1 (PSEN1) (SEQ ID NO: 5), or a variant thereof or a fragment thereof, granulin (GRN) (SEQ ID NO: 6) or a variant thereof or a fragment thereof, alpha-synuclein (SNCA) isoform 1 (SEQ ID NO: 7), isoform 2-4 (SEQ ID NO: 8), isoform 2-5 (SEQ ID NO: 9) or a variant thereof or a fragment thereof, leucine-rich repeat kinase 2 (LRRK2) (SEQ ID NO: 10) or a variant thereof or a fragment thereof, ATPase type 13A2 (ATP13A2) (SEQ ID NO: 11) or a variant thereof or a fragment thereof, superoxide dismutase 1 (SOD1) (SEQ ID NO: 12) or a variant thereof or a fragment thereof, and dynactin 1 (DCTN1) (SEQ ID NO: 13) or a variant thereof or a fragment thereof.

31. The kit according to claim 27, wherein said neurodegeneratively active protein is selected from the group consisting of: a variant of the full-length amyloid precursor protein (APP) (SEQ ID NO: 3) consisting of APP with missense mutation KM/670/671/NL or missense mutation E693G or missense mutation V717F or a fragment thereof, a variant of a microtubule-associated protein tau (MAPT) (SEQ ID NO: 4) consisting of MAPT with missense mutation L618P or a fragment thereof, a variant of full-length presenilin 1 (PSEN1) (SEQ ID NO: 5) consisting of PSEN1 with missense mutation V82L or missense mutation V96F or deletion IM 83/84 or a fragment thereof, a variant of granulin (GRN) (SEQ ID NO: 6) or a fragment thereof, a variant of alpha-synuclein (SNCA) isoform 1 (SEQ ID NO: 7), isoform 2-4 (SEQ ID NO: 8), isoform 2-5 (SEQ ID NO: 9) or a fragment thereof, a variant of leucine-rich repeat kinase 2 (LRRK2) (SEQ ID NO: 10) consisting of LRRK2 with missense mutation G2019S or a fragment thereof, a variant of ATPase type 13A2 (ATP13A2) (SEQ ID NO: 11) consisting of ATP13A2 with missense mutation G504R or a fragment thereof, a variant of superoxide dismutase 1 (SOD1) (SEQ ID NO: 12) consisting of SOD1 with missense mutation G93A or a fragment thereof, and a variant of dynactin 1 (DCTN1) (SEQ ID NO: 13) consisting of DCTN1 with missense mutation M571T or a fragment thereof.


SEQUENCE LISTING (34 sequences)

SEQ ID NO: 1 - protein, 3203 aa, Homo sapiens
MATLEKLMKAFESLKSFQQQQQQQQQQQQQQQQQQQQQQQQQQQQQQQQQQQQQQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTT

SEQ ID NO: 2 - protein, 256 aa, Artificial Sequence
  Htt171-82Q coupled with linking sequences, myc tag and His tag sequences
MATLEKLMKAFESLKSFQQQQQQQQQQQQQQQQQQQQQQQQQQQQQQQQQQQQQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLESRGPFEQKLISEEDLNMHTGHHHHHH

SEQ ID NO: 3 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 6 - protein, 593 aa, Homo sapiens
MWTLVSWVDLTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 7 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 8 - protein, 112 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 9 - protein, 126 aa, Homo sapiens
MDVFMKGLSKAKEGVVAAAEKTKQGVAEAAGKTKEGVLYVVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 10 - protein, 2527 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 11 - protein, 1180 aa, Homo sapiens
MSADSSPLVGSTPTGYGTLTIGTSIDPLSSSVSSVRLSGYCGSPWRVIGYHVVVWMMAGIPLLLFRWKPLWGVRLRLRPCNLAHAETLVIEIRDKEDSSWQLFTVQVQTEAIGEGSLEPSPQSQAEDGRSQAAVGAVPEGAWKDTAQLHKSEEAVSVGQKRVLRYYLFQGQRYIWIETQQAFYQVSLLDHGRSCDDVHRSRHGLSLQDQMVRKAIYGPNVISIPVKSYPQLLVDEALNPYYGFQAFSIALWLADHYYWYALCIFLISSISICLSLYKTRKQSQTLRDMVKLSMRVCVCRPGGEEEWVDSSELVPGDCLVLPQEGGLMPCDAALVAGECMVNESSLTGESIPVLKTALPEGLGPYCAETHRRHTLFCGTLILQARAYVGPHVLAVVTRTGFCTAKGGLVSSILHPRPINFKFYKHSMKFVAALSVLALLGTIYSIFILYRNRVPLNEIVIRALDLVTVVVPPALPAAMTVCTLYAQSRLRRQGIFCIHPLRINLGGKLQLVCFDKTGTLTEDGLDVMGVVPLKGQAFLPLVPEPRRLPVGPLLRALATCHALSRLQDTPVGDPMDLKMVESTGWVLEEEPAADSAFGTQVLAVMRPPLWEPQLQAMEEPPVPVSVLHRFPFSSALQRMSVVVAWPGATQPEAYVKGSPELVAGLCNPETVPTDFAQMLQSYTAAGYRVVALASKPLPTVPSLEAAQQLTRDTVEGDLSLLGLLVMRNLLKPQTTPVIQALRRTRIRAVMVTGDNLQTAVTVARGCGMVAPQEHLIIVHATHPERGQPASLEFLPMESPTAVNGVKDPDQAASYTVEPDPRSRHLALSGPTFGIIVKHFPKLLPKVLVQGTVFARMAPEQKTELVCELQKLQYCVGMCGDGANDCGALKAADVGISLSQAEASVVSPFTSSMASIECVPMVIREGRCSLDTSFSVFKYMALYSLTQFISVLILYTINTNLGDLQFLAIDLVITTTVAVLMSRTGPALVLGRVRPPGALLSVPVLSSLLLQMVLVTGVQLGGYFLTLAQPWFVPLNRTVAAPDNLPNYENTVVFSLSSFQYLILAAAVSKGAPFRRPLYTNVPFLVALALLSSVLVGLVLVPGLLQGPLALRNITDTGFKLLLLGLVTLNFVGAFMLESVLDQCLPACLRRLRPKRASKKRFKQLERELAEQPWPPLPAGPLR

SEQ ID NO: 12 - protein, 154 aa, Homo sapiens
MATKAVCVLKGDGPVQGIINFEQKESNGPVKVWGSIKGLTEGLHGFHVHEFGDNTAGCTSAGPHFNPLSRKHGGPKDEERHVGDLGNVTADKDAVADVSIEDSVISLSGDHCIIGRTLVVHEKADDLGKGGNEESTKTGNAGSRLACGVIGIAQ

SEQ ID NO: 13 - protein, 1278 aa, Homo sapiens
MAQSKRHVYSRTPSGSRMSAEASARPLRVGSRVEVIGKGHRGTVAYVGATLFATGKWVGVILDEAKGKNDGTVQGRKYFTCDEGHGIFVRQSQIQVFEDGADTTSPETPDSSASKVLKREGTDTTAKTSKLRGLKPKKAPTARKTTTRRPKPTRPASTGVAGASSSLGPSGSASAGELSSSEPSTPAQTPLAAPIIPTPVLTSPGAVPPLPSPSKEEEGLRAQVRDLEEKLETLRLKRAEDKAKLKELEKHKIQLEQVQEWKSKMQEQQADLQRRLKEARKEAKEALEAKERYMEEMADTADAIEMATLDKEMAEERAESLQQEVEALKERVDELTTDLEILKAEIEEKGSDGAASSYQLKQLEEQNARLKDALVRMRDLSSSEKQEHVKLQKLMEKKNQELEVVRQQRERLQEELSQAESTIDELKEQVDAALGAEEMVEMLTDRNLNLEEKVRELRETVGDLEAMNEMNDELQENARETELELREQLDMAGARVREAQKRVEAAQETVADYQQTIKKYRQLTAHLQDVNRELTNQQEASVERQQQPPPETFDFKIKFAETKAHAKAIETELRQMEVAQANRHMSLLTAFMPDSFLRPGGDHDCVLVLLLMPRLICKAELIRKQAQEKFELSENCSERPGLRGAAGEQLSFAAGLVYSLSLLQATLHRYEHALSQCSVDVYKKVGSLYPEMSAHERSLDFLIELLHKDQLDETVNVEPLTKAIKYYQHLYSIHLAEQPEDCTMQLADHIKFTQSALDCMSVEVGRLRAFLQGGQEATDIALLLRDLETSCSDIRQFCKKIRRRMPGTDAPGIPAALAFGPQVSDTLLDCRKHLTWVVAVLQEVAAAAAQLIAPLAENEGLLVAALEELAFKASEQIYGTPSSSPYECLRQSCNILISTMNKLATAMQEGEYDAERPPSKPPPVELRAAALRAEITDAEGLGLKLEDRETVIKELKKSLKIKGEELSEANVRLSLLEKKLDSAAKDADERIEKVQTRLEETQALLRKKEKEFEETMDALQADIDQLEAEKAELKQRLNSQSKRTIEGLRGPPPSGIATLVSGIAGEEQQRGAIPGQAPGSVPGPGLVKDSPLLLQQISAMRLHISQLQHENSILKGAQMKASLASLPPLHVAKLSHEGPGSELPAGALYRKTSQLLETLNQLSTHTHVVDITRTSPAAKSPSAQLMEQVAQLKSLSDTVEKLKDEVLKETVSQRPGATVPTDFATFPSSAFLRAKEEQQDDTVYMGKVTFSCAAGFGQRHRLVLTQEQLHQLHSRLIS

SEQ ID NO: 14 - DNA, 771 bp, Artificial Sequence
  sequence encoding Htt171-82Q + linking sequences + myc tag + 6X His tag
atggcgaccctggaaaagctgatgaaggccttcgagtccctcaagtccttccagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagtctagagggcccttcgaacaaaaactcatctcagaagaggatctgaatatgcataccggtcatcatcaccatcaccattga

SEQ ID NO: 15 - DNA, 9373 bp, Artificial Sequence
  Plasmid encoding the first 171 amino acids of mutated htt (htt171-82Q)
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgaccatcgatggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcaattctctagagatatcgtcgatggcgaccctggaaaagctgatgaaggccttcgagtccctcaagtccttccagcagcagcagcagcagcagcagcagcagcagcagcagcagcagcagcaacagccgccaccgccgccgccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagtctagagggcccttcgaacaaaaactcatctcagaagaggatctgaatatgcataccggtcatcatcaccatcaccattgagttttcgagtgagagaagattttcagcctgatacagattaaaatcgtcgagggaattgatcctctagagtcgacctgcaggcatgcaagctaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattagcttgttaacatcgatggaattcgagctcggtacctttaaaaccaatgacttacaaggcagctgtaaatcttagccactttttaaaagaaaaggggggactggaagggctaattcactcccaacgaaaacaaaatctgctttttgcttgtactgggtctctctggttagaccaaatctgagcctgggagctctctggctaactagggaacccactgcttaagcctcaataaagcttgccttgagtgcttcaagtagtgtgtgcccgtctgttgtgtgactctggtaactagagatccctcaaacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagagcttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtgtggatcaactggataactcaagctaaccaaaatcatcccaaacttcccaccccataccctattaccactgccaaattacctgtggtttcatttactctaaacctgtgattcctctgaattattttcattttaaagaaattgtatttgttaaatatgtactacaaacttagtagt

SEQ ID NO: 16 - DNA, 9558 bp, Artificial Sequence
  Plasmid encoding the first 171 amino acids of wild-type htt (htt171-18Q)
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgaccatcgatggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcaattctctagagatatcgtcgacatggcgaccctggaaaagctgatgaaggccttcgagtccctcaagtccttccagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagtctagagggcccttcgaacaaaaactcatctcagaagaggatctgaatatgcataccggtcatcatcaccatcaccattgagttttcgagtgagagaagattttcagcctgatacagattaaaatcgtcgagggaattgatcctctagagtcgacctgcaggcatgcaagctaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattagcttgttaacatcgatggaattcgagctcggtacctttaaaaccaatgacttacaaggcagctgtaaatcttagccactttttaaaagaaaaggggggactggaagggctaattcactcccaacgaaaacaaaatctgctttttgcttgtactgggtctctctggttagaccaaatctgagcctgggagctctctggctaactagggaacccactgcttaagcctcaataaagcttgccttgagtgcttcaagtagtgtgtgcccgtctgttgtgtgactctggtaactagagatccctcaaacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagagcttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtgtggatcaactggataactcaagctaaccaaaatcatcccaaacttcccaccccataccctattaccactgccaaattacctgtggtttcatttactctaaacctgtgattcctctgaattattttcattttaaagaaattgtatttgttaaatatgtactacaaacttagtagt

SEQ ID NO: 17 - DNA, 20 bp, Artificial Sequence
  forward primer for amplifying a fragment of exon I of BDNF
tgttggggagacgagatttt

SEQ ID NO: 18 - DNA, 20 bp, Artificial Sequence
  reverse primer for amplifying a fragment of exon I of BDNF
cgtggacgtttgcttctttc

SEQ ID NO: 19 - DNA, 21 bp, Artificial Sequence
  forward primer for amplifying a fragment of exon IIa of BDNF
tacttcatccagttccaccag

SEQ ID NO: 20 - DNA, 17 bp, Artificial Sequence
  reverse primer for amplifying a fragment of exon IIa of BDNF
caagttgccttgtccgt

SEQ ID NO: 21 - DNA, 18 bp, Artificial Sequence
  forward primer for amplifying a fragment of exon IIb of BDNF
aagctccggttccaccag

SEQ ID NO: 22 - DNA, 17 bp, Artificial Sequence
  reverse primer for amplifying a fragment of exon IIb of BDNF
tgcttctttcatgggcg

SEQ ID NO: 23 - DNA, 19 bp, Artificial Sequence
  forward primer for amplifying a fragment of exon IIc of BDNF
gtggtgtaagccgcaaaga

SEQ ID NO: 24 - DNA, 20 bp, Artificial Sequence
  reverse primer for amplifying a fragment of exon IIc of BDNF
cgtggacgtttgcttctttc

SEQ ID NO: 25 - DNA, 19 bp, Artificial Sequence
  forward primer for amplifying a fragment of exon III of BDNF
ctgagactgcgctccactc

SEQ ID NO: 26 - DNA, 20 bp, Artificial Sequence
  reverse primer for amplifying a fragment of exon III of BDNF
gtggacgtttgcttctttca

SEQ ID NO: 27 - DNA, 20 bp, Artificial Sequence
  forward primer for amplifying a fragment of exon VI of BDNF
gatccgagagctttgtgtgg

SEQ ID NO: 28 - DNA, 20 bp, Artificial Sequence
  reverse primer for amplifying a fragment of exon VI of BDNF
gtggacgtttgcttctttca

SEQ ID NO: 29 - DNA, 32 bp, Artificial Sequence
  forward primer for amplifying a fragment of exon IV of BDNF
cgccatgcaatttccactatcaataatttaac

SEQ ID NO: 30 - DNA, 30 bp, Artificial Sequence
  reverse primer for amplifying a fragment of exon IV of BDNF
gtttactttgacaagtagtgactgaaaaag

SEQ ID NO: 31 - DNA, 15 bp, Artificial Sequence
  universal forward primer for PCR
tggatgccgcaaaca

SEQ ID NO: 32 - DNA, 20 bp, Artificial Sequence
  universal reverse primer for PCR
ccgggactttctccaggact

SEQ ID NO: 33 - DNA, 19 bp, Artificial Sequence
  forward primer for amplifying a fragment of beta-actin
aggcatcctgaccctgaag

SEQ ID NO: 34 - DNA, 20 bp, Artificial Sequence
  reverse primer for amplifying a fragment of beta-actin
gctcattgtagaaagtgtgg